SEQUENCE LISTING (225 sequences)

SEQ ID NO: 1 - DNA, 16 bp, Artificial Sequence
  Description of Artificial Sequence Synthetic oligonucleotide
cactccggacgcccct

SEQ ID NO: 2 - DNA, 20 bp, Artificial Sequence
  Description of Artificial Sequence Synthetic oligonucleotide
gatgacgttgctcacggctt

SEQ ID NO: 3 - DNA, 22 bp, Artificial Sequence
  Description of Artificial Sequence Synthetic oligonucleotide
tcgccccaacggtctcttggac

SEQ ID NO: 4 - DNA, 22 bp, Artificial Sequence
  Description of Artificial Sequence Synthetic oligonucleotide
ctcatttggaattttgccgatt

SEQ ID NO: 5 - DNA, 22 bp, Artificial Sequence
  Description of Artificial Sequence Synthetic oligonucleotide
ccgagtgaagatccccttttta

SEQ ID NO: 6 - DNA, 26 bp, Artificial Sequence
  Description of Artificial Sequence Synthetic oligonucleotide
tgaacagtcaccgacgagagtgctgg

SEQ ID NO: 7 - DNA, 20 bp, Artificial Sequence
  Description of Artificial Sequence Synthetic oligonucleotide
atggacattgaagcatatct

SEQ ID NO: 8 - DNA, 20 bp, Artificial Sequence
  Description of Artificial Sequence Synthetic oligonucleotide
tgtggttatcttggaaattg

SEQ ID NO: 9 - DNA, 17 bp, Artificial Sequence
  Description of Artificial Sequence Synthetic oligonucleotide
ggaacaaattggacttg

SEQ ID NO: 10 - DNA, 20 bp, Artificial Sequence
  Description of Artificial Sequence Synthetic oligonucleotide
gcagagtgattcatgctaga

SEQ ID NO: 11 - DNA, 20 bp, Artificial Sequence
  Description of Artificial Sequence Synthetic oligonucleotide
actcacagcagagggcaaag

SEQ ID NO: 12 - DNA, 20 bp, Artificial Sequence
  Description of Artificial Sequence Synthetic oligonucleotide
cgaagcagtatggtgttctg

SEQ ID NO: 13 - DNA, 20 bp, Artificial Sequence
  Description of Artificial Sequence Synthetic oligonucleotide
tgcacgctacccaccaggcc

SEQ ID NO: 14 - DNA, 20 bp, Artificial Sequence
  Description of Artificial Sequence Synthetic oligonucleotide
tgcacgctactcaccaggcc

SEQ ID NO: 15 - DNA, 21 bp, Artificial Sequence
  Description of Artificial Sequence Synthetic oligonucleotide
gtgatcctggcttgacaagag

SEQ ID NO: 16 - DNA, 19 bp, Artificial Sequence
  Description of Artificial Sequence Synthetic oligonucleotide
cggaaatctgtctctgtcc

SEQ ID NO: 17 - DNA, 21 bp, Artificial Sequence
  Description of Artificial Sequence Synthetic oligonucleotide
gcgcgaggcgctggtgaccca

SEQ ID NO: 18 - DNA, 21 bp, Artificial Sequence
  Description of Artificial Sequence Synthetic oligonucleotide
gcgcgaggcgatggtgaccca

SEQ ID NO: 19 - DNA, 21 bp, Artificial Sequence
  Description of Artificial Sequence Synthetic oligonucleotide
gtgcccatcacccagatcctg

SEQ ID NO: 20 - DNA, 21 bp, Artificial Sequence
  Description of Artificial Sequence Synthetic oligonucleotide
gtgcccatcatccagatcctg

SEQ ID NO: 21 - DNA, 21 bp, Artificial Sequence
  Description of Artificial Sequence Synthetic oligonucleotide
tcctgggtttcgggccgcgtt

SEQ ID NO: 22 - DNA, 21 bp, Artificial Sequence
  Description of Artificial Sequence Synthetic oligonucleotide
tcctgggttttgggccgcgtt

SEQ ID NO: 23 - DNA, 19 bp, Artificial Sequence
  Description of Artificial Sequence Synthetic oligonucleotide
tggatggtggggctaatgc

SEQ ID NO: 24 - DNA, 21 bp, Artificial Sequence
  Description of Artificial Sequence Synthetic oligonucleotide
tatgcaaatcctgctcttccg

SEQ ID NO: 25 - DNA, 21 bp, Artificial Sequence
  Description of Artificial Sequence Synthetic oligonucleotide
gcccgcgtggggcgagcagag

SEQ ID NO: 26 - DNA, 21 bp, Artificial Sequence
  Description of Artificial Sequence Synthetic oligonucleotide
gcccgcgtggcgcgagcagag

SEQ ID NO: 27 - DNA, 19 bp, Artificial Sequence
  Description of Artificial Sequence Synthetic oligonucleotide
cgcttctccgtgtccacct

SEQ ID NO: 28 - DNA, 19 bp, Artificial Sequence
  Description of Artificial Sequence Synthetic oligonucleotide
cgcttctccgtctccacct

SEQ ID NO: 29 - DNA, 21 bp, Artificial Sequence
  Description of Artificial Sequence Synthetic oligonucleotide
gctggagcagtgggtgaccga

SEQ ID NO: 30 - DNA, 20 bp, Artificial Sequence
  Description of Artificial Sequence Synthetic oligonucleotide
gctggagcaggggtgaccga

SEQ ID NO: 31 - DNA, 21 bp, Artificial Sequence
  Description of Artificial Sequence Synthetic oligonucleotide
gccttcgccaaccactccggt

SEQ ID NO: 32 - DNA, 21 bp, Artificial Sequence
  Description of Artificial Sequence Synthetic oligonucleotide
gccttcgccgaccactccggt

SEQ ID NO: 33 - DNA, 21 bp, Artificial Sequence
  Description of Artificial Sequence Synthetic oligonucleotide
caaccactccggtgggtgatg

SEQ ID NO: 34 - DNA, 21 bp, Artificial Sequence
  Description of Artificial Sequence Synthetic oligonucleotide
caaccactcctgtgggtgatg

SEQ ID NO: 35 - DNA, 21 bp, Artificial Sequence
  Description of Artificial Sequence Synthetic oligonucleotide
gctcaggagggactgaaggag

SEQ ID NO: 36 - DNA, 21 bp, Artificial Sequence
  Description of Artificial Sequence Synthetic oligonucleotide
gctcaggaggaactgaaggag

SEQ ID NO: 37 - DNA, 20 bp, Artificial Sequence
  Description of Artificial Sequence Synthetic oligonucleotide
atgggcagaagggcacaaag

SEQ ID NO: 38 - DNA, 20 bp, Artificial Sequence
  Description of Artificial Sequence Synthetic oligonucleotide
ttgtccaagagaccgttggg

SEQ ID NO: 39 - DNA, 21 bp, Artificial Sequence
  Description of Artificial Sequence Synthetic oligonucleotide
gcatctcccacccccaggacg

SEQ ID NO: 40 - DNA, 21 bp, Artificial Sequence
  Description of Artificial Sequence Synthetic oligonucleotide
gcatctcccacccccaagacg

SEQ ID NO: 41 - DNA, 20 bp, Artificial Sequence
  Description of Artificial Sequence Synthetic oligonucleotide
acttgtccaggtgaacgcag

SEQ ID NO: 42 - DNA, 20 bp, Artificial Sequence
  Description of Artificial Sequence Synthetic oligonucleotide
ctgacctccaattctgcacc

SEQ ID NO: 43 - DNA, 21 bp, Artificial Sequence
  Description of Artificial Sequence Synthetic oligonucleotide
aactgagcacaggatgacctg

SEQ ID NO: 44 - DNA, 20 bp, Artificial Sequence
  Description of Artificial Sequence Synthetic oligonucleotide
aactgagcacggatgacctg

SEQ ID NO: 45 - DNA, 19 bp, Artificial Sequence
  Description of Artificial Sequence Synthetic oligonucleotide
ttctgtcccgagtatgctc

SEQ ID NO: 46 - DNA, 20 bp, Artificial Sequence
  Description of Artificial Sequence Synthetic oligonucleotide
acaggcacctgctgagaaag

SEQ ID NO: 47 - DNA, 21 bp, Artificial Sequence
  Description of Artificial Sequence Synthetic oligonucleotide
tgagaacctgcgcatagtggt

SEQ ID NO: 48 - DNA, 21 bp, Artificial Sequence
  Description of Artificial Sequence Synthetic oligonucleotide
tgagaacctgtgcatagtggt

SEQ ID NO: 49 - DNA, 22 bp, Artificial Sequence
  Description of Artificial Sequence Synthetic oligonucleotide
atcctacatccggatgtgcagc

SEQ ID NO: 50 - DNA, 22 bp, Artificial Sequence
  Description of Artificial Sequence Synthetic oligonucleotide
atcctacatctggatgtgcagc

SEQ ID NO: 51 - DNA, 18 bp, Artificial Sequence
  Description of Artificial Sequence Synthetic oligonucleotide
aggcaagaaggagtgtca

SEQ ID NO: 52 - DNA, 19 bp, Artificial Sequence
  Description of Artificial Sequence Synthetic oligonucleotide
tcagtgtggtggcattgag

SEQ ID NO: 53 - DNA, 21 bp, Artificial Sequence
  Description of Artificial Sequence Synthetic oligonucleotide
cgtccccctgggtgtgaccca

SEQ ID NO: 54 - DNA, 21 bp, Artificial Sequence
  Description of Artificial Sequence Synthetic oligonucleotide
cgtccccctgagtgtgaccca

SEQ ID NO: 55 - DNA, 20 bp, Artificial Sequence
  Description of Artificial Sequence Synthetic oligonucleotide
cagcatcctagagtccgtcc

SEQ ID NO: 56 - DNA, 20 bp, Artificial Sequence
  Description of Artificial Sequence Synthetic oligonucleotide
acaggcacctgctgagaaag

SEQ ID NO: 57 - DNA, 21 bp, Artificial Sequence
  Description of Artificial Sequence Synthetic oligonucleotide
acctgtcatcggtgctgaagg

SEQ ID NO: 58 - DNA, 21 bp, Artificial Sequence
  Description of Artificial Sequence Synthetic oligonucleotide
acctgtcatcagtgctgaagg

SEQ ID NO: 59 - DNA, 20 bp, Artificial Sequence
  Description of Artificial Sequence Synthetic oligonucleotide
cttccaccccgaacacttcc

SEQ ID NO: 60 - DNA, 20 bp, Artificial Sequence
  Description of Artificial Sequence Synthetic oligonucleotide
cttccacccccaacacttcc

SEQ ID NO: 61 - DNA, 19 bp, Artificial Sequence
  Description of Artificial Sequence Synthetic oligonucleotide
agtcttgcaggggtatcac

SEQ ID NO: 62 - DNA, 20 bp, Artificial Sequence
  Description of Artificial Sequence Synthetic oligonucleotide
tctgctcagcctcaacgtac

SEQ ID NO: 63 - DNA, 22 bp, Artificial Sequence
  Description of Artificial Sequence Synthetic oligonucleotide
tgagcccatccccccctatgag

SEQ ID NO: 64 - DNA, 20 bp, Artificial Sequence
  Description of Artificial Sequence Synthetic oligonucleotide
tgaccccatccccctatgag

SEQ ID NO: 65 - DNA, 21 bp, Artificial Sequence
  Description of Artificial Sequence Synthetic oligonucleotide
ttcaagaccagcctggacaac

SEQ ID NO: 66 - DNA, 20 bp, Artificial Sequence
  Description of Artificial Sequence Synthetic oligonucleotide
gtgccaccacgtctagcttt

SEQ ID NO: 67 - DNA, 21 bp, Artificial Sequence
  Description of Artificial Sequence Synthetic oligonucleotide
ttggaagaacccggtctctac

SEQ ID NO: 68 - DNA, 21 bp, Artificial Sequence
  Description of Artificial Sequence Synthetic oligonucleotide
ttggaagaacgcggtctctac

SEQ ID NO: 69 - DNA, 26 bp, Artificial Sequence
  Description of Artificial Sequence Synthetic oligonucleotide
agaaagatccaagaggagctagacac

SEQ ID NO: 70 - DNA, 20 bp, Artificial Sequence
  Description of Artificial Sequence Synthetic oligonucleotide
tgggatctgtcagagagccg

SEQ ID NO: 71 - DNA, 18 bp, Artificial Sequence
  Description of Artificial Sequence Synthetic oligonucleotide
tggcaggtcacggcggcc

SEQ ID NO: 72 - DNA, 20 bp, Artificial Sequence
  Description of Artificial Sequence Synthetic oligonucleotide
atcgcctctgacccagcttc

SEQ ID NO: 73 - DNA, 19 bp, Artificial Sequence
  Description of Artificial Sequence Synthetic oligonucleotide
ctcatcgctactctcaggg

SEQ ID NO: 74 - DNA, 20 bp, Artificial Sequence
  Description of Artificial Sequence Synthetic oligonucleotide
acgtcattggtgccatgtgc

SEQ ID NO: 75 - DNA, 23 bp, Artificial Sequence
  Description of Artificial Sequence Synthetic oligonucleotide
accaggtatcctgatgtgcagac

SEQ ID NO: 76 - DNA, 16 bp, Artificial Sequence
  Description of Artificial Sequence Synthetic oligonucleotide
acggtccctccccacg

SEQ ID NO: 77 - DNA, 25 bp, Artificial Sequence
  Description of Artificial Sequence Synthetic oligonucleotide
cgagtgcaggcagaattggatcagg

SEQ ID NO: 78 - DNA, 20 bp, Artificial Sequence
  Description of Artificial Sequence Synthetic oligonucleotide
actacctcatccccaagggc

SEQ ID NO: 79 - DNA, 26 bp, Artificial Sequence
  Description of Artificial Sequence Synthetic oligonucleotide
ttggattaggaaattctttgtcatca

SEQ ID NO: 80 - DNA, 32 bp, Artificial Sequence
  Description of Artificial Sequence Synthetic oligonucleotide
caaccataatggcattactgacttccgtgcta

SEQ ID NO: 81 - DNA, 22 bp, Artificial Sequence
  Description of Artificial Sequence Synthetic oligonucleotide
tttctcagcaggaaaacggatt

SEQ ID NO: 82 - DNA, 24 bp, Artificial Sequence
  Description of Artificial Sequence Synthetic oligonucleotide
ggtcaggaataaaaacagctccat

SEQ ID NO: 83 - DNA, 21 bp, Artificial Sequence
  Description of Artificial Sequence Synthetic oligonucleotide
tgtgggagaagccctggcccg

SEQ ID NO: 84 - DNA, 21 bp, Artificial Sequence
  Description of Artificial Sequence Synthetic oligonucleotide
tcagcaggaaaacggatgtgt

SEQ ID NO: 85 - DNA, 23 bp, Artificial Sequence
  Description of Artificial Sequence Synthetic oligonucleotide
tggtggtcaggaataaaaacagc

SEQ ID NO: 86 - DNA, 18 bp, Artificial Sequence
  Description of Artificial Sequence Synthetic oligonucleotide
agggcctggcccgcatgg

SEQ ID NO: 87 - DNA, 21 bp, Artificial Sequence
  Description of Artificial Sequence Synthetic oligonucleotide
ctgatcaaaatggagaaggaa

SEQ ID NO: 88 - DNA, 19 bp, Artificial Sequence
  Description of Artificial Sequence Synthetic oligonucleotide
gctgcagtgattaccaagt

SEQ ID NO: 89 - DNA, 19 bp, Artificial Sequence
  Description of Artificial Sequence Synthetic oligonucleotide
aaaccaacagtctgaattc

SEQ ID NO: 90 - DNA, 16 bp, Artificial Sequence
  Description of Artificial Sequence Synthetic oligonucleotide
ttctcgggcagaggcg

SEQ ID NO: 91 - DNA, 21 bp, Artificial Sequence
  Description of Artificial Sequence Synthetic oligonucleotide
aaaatgattcccctgtccctg

SEQ ID NO: 92 - DNA, 19 bp, Artificial Sequence
  Description of Artificial Sequence Synthetic oligonucleotide
cctccccgcgttccatgcg

SEQ ID NO: 93 - DNA, 21 bp, Artificial Sequence
  Description of Artificial Sequence Synthetic oligonucleotide
tgtggggcttttatgatggtc

SEQ ID NO: 94 - DNA, 28 bp, Artificial Sequence
  Description of Artificial Sequence Synthetic oligonucleotide
ggtttgtgaagacagaataacattcttt

SEQ ID NO: 95 - DNA, 31 bp, Artificial Sequence
  Description of Artificial Sequence Synthetic oligonucleotide
tcacagatcctgacatgatcaaaacagtgct

SEQ ID NO: 96 - DNA, 12 bp, Artificial Sequence
  Description of Artificial Sequence Synthetic oligonucleotide
ttaaggttctca

SEQ ID NO: 97 - DNA, 12 bp, Artificial Sequence
  Description of Artificial Sequence Synthetic oligonucleotide
ttaaggttctcg

SEQ ID NO: 98 - DNA, 50 bp, Artificial Sequence
  Description of Artificial Sequence Synthetic oligonucleotide
acattcttgagcaccagatccgggctgttccctttgagaaccttaacatg

SEQ ID NO: 99 - DNA, 50 bp, Artificial Sequence
  Description of Artificial Sequence Synthetic oligonucleotide
acattcttgagcaccagatccgggctgttcccttcgagaaccttaacatg

SEQ ID NO: 100 - DNA, 42 bp, Artificial Sequence
  Description of Artificial Sequence Synthetic oligonucleotide
aagggaacagcccgaatctggtgctcaagaatgtcagttaat

SEQ ID NO: 101 - DNA, 9 bp, Artificial Sequence
  Description of Artificial Sequence Synthetic oligonucleotide
acccacccc

SEQ ID NO: 102 - DNA, 9 bp, Artificial Sequence
  Description of Artificial Sequence Synthetic oligonucleotide
acccaccct

SEQ ID NO: 103 - DNA, 50 bp, Artificial Sequence
  Description of Artificial Sequence Synthetic oligonucleotide
gaggctatttttgatcacattgtaagaagaaaccggggtgggtggtgtct

SEQ ID NO: 104 - DNA, 50 bp, Artificial Sequence
  Description of Artificial Sequence Synthetic oligonucleotide
gaggctatttttgatcacattgtaagaagaaaccagggtgggtggtgtct

SEQ ID NO: 105 - DNA, 42 bp, Artificial Sequence
  Description of Artificial Sequence Synthetic oligonucleotide
ggtttcttcttacaatgtgatcaaaaatagcctctaagccca

SEQ ID NO: 106 - DNA, 14 bp, Artificial Sequence
  Description of Artificial Sequence Synthetic oligonucleotide
gagggtatttttac

SEQ ID NO: 107 - DNA, 14 bp, Artificial Sequence
  Description of Artificial Sequence Synthetic oligonucleotide
gagggtatttttat

SEQ ID NO: 108 - DNA, 50 bp, Artificial Sequence
  Description of Artificial Sequence Synthetic oligonucleotide
aacatgccagtgctgtatttgttaactggagggatgtaaaaataccctcc

SEQ ID NO: 109 - DNA, 50 bp, Artificial Sequence
  Description of Artificial Sequence Synthetic oligonucleotide
aacatgccagtgctgtatttgttaactggagggatataaaaataccctcc

SEQ ID NO: 110 - DNA, 42 bp, Artificial Sequence
  Description of Artificial Sequence Synthetic oligonucleotide
atccctccagttaacaaatacagcactggcatggttcacctt

SEQ ID NO: 111 - DNA, 10 bp, Artificial Sequence
  Description of Artificial Sequence Synthetic oligonucleotide
caaggcacct

SEQ ID NO: 112 - DNA, 9 bp, Artificial Sequence
  Description of Artificial Sequence Synthetic oligonucleotide
aaggcaccg

SEQ ID NO: 113 - DNA, 50 bp, Artificial Sequence
  Description of Artificial Sequence Synthetic oligonucleotide
cagcctctagaattaatttctgggaaggatcagcctcaggtgccttgcat

SEQ ID NO: 114 - DNA, 50 bp, Artificial Sequence
  Description of Artificial Sequence Synthetic oligonucleotide
cagcctctagaattaatttctgggaaggatcagcctccggtgccttgcat

SEQ ID NO: 115 - DNA, 42 bp, Artificial Sequence
  Description of Artificial Sequence Synthetic oligonucleotide
gaggctgatccttcccagaaagtaattctagaggctgccaca

SEQ ID NO: 116 - DNA, 12 bp, Artificial Sequence
  Description of Artificial Sequence Synthetic oligonucleotide
aatatactgctc

SEQ ID NO: 117 - DNA, 12 bp, Artificial Sequence
  Description of Artificial Sequence Synthetic oligonucleotide
aatatactgctt

SEQ ID NO: 118 - DNA, 50 bp, Artificial Sequence
  Description of Artificial Sequence Synthetic oligonucleotide
gaggaatctggtacctggaccaaatcaggagagagcagtatattacaaac

SEQ ID NO: 119 - DNA, 50 bp, Artificial Sequence
  Description of Artificial Sequence Synthetic oligonucleotide
gaggaatctggtacctggaccaaatcaggagaaagcagtatattacaaac

SEQ ID NO: 120 - DNA, 42 bp, Artificial Sequence
  Description of Artificial Sequence Synthetic oligonucleotide
tctcctgatttggtccaggtaccagattcctctctcttctgt

SEQ ID NO: 121 - DNA, 13 bp, Artificial Sequence
  Description of Artificial Sequence Synthetic oligonucleotide
agttttaaactcg

SEQ ID NO: 122 - DNA, 13 bp, Artificial Sequence
  Description of Artificial Sequence Synthetic oligonucleotide
agttttaaactca

SEQ ID NO: 123 - DNA, 50 bp, Artificial Sequence
  Description of Artificial Sequence Synthetic oligonucleotide
aaattcaattataaagacaatacagatctggtcgagtttaaaactctcac

SEQ ID NO: 124 - DNA, 50 bp, Artificial Sequence
  Description of Artificial Sequence Synthetic oligonucleotide
aaattcaattataaagacaatacagatctggttgagtttaaaactctcac

SEQ ID NO: 125 - DNA, 42 bp, Artificial Sequence
  Description of Artificial Sequence Synthetic oligonucleotide
acgagatctgtattgtctttataattgaattttctataggtg

SEQ ID NO: 126 - DNA, 11 bp, Artificial Sequence
  Description of Artificial Sequence Synthetic oligonucleotide
gaagtgctgaa

SEQ ID NO: 127 - DNA, 11 bp, Artificial Sequence
  Description of Artificial Sequence Synthetic oligonucleotide
gaagtgctgag

SEQ ID NO: 128 - DNA, 51 bp, Artificial Sequence
  Description of Artificial Sequence Synthetic oligonucleotide
acgagatttctccccaaggaaatcttaaatatatttttcagcacttcttca

SEQ ID NO: 129 - DNA, 51 bp, Artificial Sequence
  Description of Artificial Sequence Synthetic oligonucleotide
acgagatttctccccaaggaaatcttaaatatatttctcagcacttcttca

SEQ ID NO: 130 - DNA, 40 bp, Artificial Sequence
  Description of Artificial Sequence Synthetic oligonucleotide
aaatatctttaagatttccttggggagacatctcgtgccc

SEQ ID NO: 131 - DNA, 13 bp, Artificial Sequence
  Description of Artificial Sequence Synthetic oligonucleotide
gtaagaagaaacc

SEQ ID NO: 132 - DNA, 13 bp, Artificial Sequence
  Description of Artificial Sequence Synthetic oligonucleotide
gtaagaagaaact

SEQ ID NO: 133 - DNA, 50 bp, Artificial Sequence
  Description of Artificial Sequence Synthetic oligonucleotide
cagaagttgattgacctggagacaccacccaccccggtttcttcttacaa

SEQ ID NO: 134 - DNA, 50 bp, Artificial Sequence
  Description of Artificial Sequence Synthetic oligonucleotide
cagaagttgattgacctggagacaccacccaccccagtttcttcttacaa

SEQ ID NO: 135 - DNA, 42 bp, Artificial Sequence
  Description of Artificial Sequence Synthetic oligonucleotide
ggggtgggtggtgtctccaggtcaatcaacttctgtactggg

SEQ ID NO: 136 - DNA, 10 bp, Artificial Sequence
  Description of Artificial Sequence Synthetic oligonucleotide
aggtgaccat

SEQ ID NO: 137 - DNA, 10 bp, Artificial Sequence
  Description of Artificial Sequence Synthetic oligonucleotide
aggtgaccac

SEQ ID NO: 138 - DNA, 51 bp, Artificial Sequence
  Description of Artificial Sequence Synthetic oligonucleotide
ccagacccagcatcgacaatgtaattcctgccgtcaatggtcacctgcagg

SEQ ID NO: 139 - DNA, 51 bp, Artificial Sequence
  Description of Artificial Sequence Synthetic oligonucleotide
ccagacccagcatcgacaatgtaattcctgccgtcagtggtcacctgcagg

SEQ ID NO: 140 - DNA, 39 bp, Artificial Sequence
  Description of Artificial Sequence Synthetic oligonucleotide
tgacggcaggaattacattgtcgatgctgggtctggaag

SEQ ID NO: 141 - DNA, 11 bp, Artificial Sequence
  Description of Artificial Sequence Synthetic oligonucleotide
aatctggtacc

SEQ ID NO: 142 - DNA, 11 bp, Artificial Sequence
  Description of Artificial Sequence Synthetic oligonucleotide
aatctggtact

SEQ ID NO: 143 - DNA, 51 bp, Artificial Sequence
  Description of Artificial Sequence Synthetic oligonucleotide
tgtttgtaatatactgctctctcctgatttggtccaggtaccagattcctc

SEQ ID NO: 144 - DNA, 51 bp, Artificial Sequence
  Description of Artificial Sequence Synthetic oligonucleotide
tgtttgtaatatactgctctctcctgatttggtccaagtaccagattcctc

SEQ ID NO: 145 - DNA, 42 bp, Artificial Sequence
  Description of Artificial Sequence Synthetic oligonucleotide
tggaccaaatcaggagagagcagtatattacaaacaaagaat

SEQ ID NO: 146 - DNA, 10 bp, Artificial Sequence
  Description of Artificial Sequence Synthetic oligonucleotide
ttgaacctcg

SEQ ID NO: 147 - DNA, 10 bp, Artificial Sequence
  Description of Artificial Sequence Synthetic oligonucleotide
ttgaacctca

SEQ ID NO: 148 - DNA, 51 bp, Artificial Sequence
  Description of Artificial Sequence Synthetic oligonucleotide
aggtatgtattcatagactcaaaatcttcaattgttcgaggttcaagcgta

SEQ ID NO: 149 - DNA, 51 bp, Artificial Sequence
  Description of Artificial Sequence Synthetic oligonucleotide
aggtatgtattcatagactcaaaatcttcaattgtttgaggttcaagcgta

SEQ ID NO: 150 - DNA, 31 bp, Artificial Sequence
  Description of Artificial Sequence Synthetic oligonucleotide
aacaattgaagattttgagtctatgaataca

SEQ ID NO: 151 - DNA, 9 bp, Artificial Sequence
  Description of Artificial Sequence Synthetic oligonucleotide
cgtgcccaa

SEQ ID NO: 152 - DNA, 9 bp, Artificial Sequence
  Description of Artificial Sequence Synthetic oligonucleotide
cgtgcccac

SEQ ID NO: 153 - DNA, 51 bp, Artificial Sequence
  Description of Artificial Sequence Synthetic oligonucleotide
tccttattctaaatagtaagggatccatcaccaggtttgggcacgagattt

SEQ ID NO: 154 - DNA, 51 bp, Artificial Sequence
  Description of Artificial Sequence Synthetic oligonucleotide
tccttattctaaatagtaagggatccatcaccaggtgtgggcacgagattt

SEQ ID NO: 155 - DNA, 41 bp, Artificial Sequence
  Description of Artificial Sequence Synthetic oligonucleotide
acctggtgatggatcccttactatttagaataaggaacaaa

SEQ ID NO: 156 - DNA, 10 bp, Artificial Sequence
  Description of Artificial Sequence Synthetic oligonucleotide
ctggtgatgg

SEQ ID NO: 157 - DNA, 10 bp, Artificial Sequence
  Description of Artificial Sequence Synthetic oligonucleotide
ctggtgatga

SEQ ID NO: 158 - DNA, 50 bp, Artificial Sequence
  Description of Artificial Sequence Synthetic oligonucleotide
ggagaaatctcgtgcccaaacctggtgatggatcccttactatttagaat

SEQ ID NO: 159 - DNA, 50 bp, Artificial Sequence
  Description of Artificial Sequence Synthetic oligonucleotide
ggagaaatctcgtgcccaaacctggtgatgaatcccttactatttagaat

SEQ ID NO: 160 - DNA, 42 bp, Artificial Sequence
  Description of Artificial Sequence Synthetic oligonucleotide
atcccttactatttagaataaggaacaaaataaacccttgtg

SEQ ID NO: 161 - DNA, 10 bp, Artificial Sequence
  Description of Artificial Sequence Synthetic oligonucleotide
aacggaagac

SEQ ID NO: 162 - DNA, 10 bp, Artificial Sequence
  Description of Artificial Sequence Synthetic oligonucleotide
aacggaagat

SEQ ID NO: 163 - DNA, 50 bp, Artificial Sequence
  Description of Artificial Sequence Synthetic oligonucleotide
aatttctgggaaggatcagcctcaggtgccttgtgtcttccgtttgacgg

SEQ ID NO: 164 - DNA, 34 bp, Artificial Sequence
  Description of Artificial Sequence Synthetic oligonucleotide
acaaggcacctgaggctgatccttcccagaaatt

SEQ ID NO: 165 - DNA, 13 bp, Artificial Sequence
  Description of Artificial Sequence Synthetic oligonucleotide
gagtagatttttc

SEQ ID NO: 166 - DNA, 13 bp, Artificial Sequence
  Description of Artificial Sequence Synthetic oligonucleotide
gagtagatttttt

SEQ ID NO: 167 - DNA, 50 bp, Artificial Sequence
  Description of Artificial Sequence Synthetic oligonucleotide
aatttcttcattctgatctcctagaagacagcaaataccgaaaaatctac

SEQ ID NO: 168 - DNA, 39 bp, Artificial Sequence
  Description of Artificial Sequence Synthetic oligonucleotide
ggtatttgctgtcttctaggagatcagaatgaagaaatt

SEQ ID NO: 169 - DNA, 10 bp, Artificial Sequence
  Description of Artificial Sequence Synthetic oligonucleotide
acatctccat

SEQ ID NO: 170 - DNA, 10 bp, Artificial Sequence
  Description of Artificial Sequence Synthetic oligonucleotide
acatctccag

SEQ ID NO: 171 - DNA, 52 bp, Artificial Sequence
  Description of Artificial Sequence Synthetic oligonucleotide
gtctgcaaggaacaaaatgatttactagtaaacacagatgatggagatgtct

SEQ ID NO: 172 - DNA, 42 bp, Artificial Sequence
  Description of Artificial Sequence Synthetic oligonucleotide
catctgtgtgtactagtaaatcattttgttccttgcagaccc

SEQ ID NO: 173 - DNA, 13 bp, Artificial Sequence
  Description of Artificial Sequence Synthetic oligonucleotide
tttctatttcttc

SEQ ID NO: 174 - DNA, 13 bp, Artificial Sequence
  Description of Artificial Sequence Synthetic oligonucleotide
tttctatttcttt

SEQ ID NO: 175 - DNA, 50 bp, Artificial Sequence
  Description of Artificial Sequence Synthetic oligonucleotide
gatctaatagagttcaagactctgagtgaggaagaaatagaaaaagtgct

SEQ ID NO: 176 - DNA, 30 bp, Artificial Sequence
  Description of Artificial Sequence Synthetic oligonucleotide
ctcactcagagtcttgaactctattagatc

SEQ ID NO: 177 - DNA, 13 bp, Artificial Sequence
  Description of Artificial Sequence Synthetic oligonucleotide
taaaagacattta

SEQ ID NO: 178 - DNA, 13 bp, Artificial Sequence
  Description of Artificial Sequence Synthetic oligonucleotide
taaaagacatttt

SEQ ID NO: 179 - DNA, 50 bp, Artificial Sequence
  Description of Artificial Sequence Synthetic oligonucleotide
cacaaaccttttcaaataataataataataataataataaatgtctttta

SEQ ID NO: 180 - DNA, 37 bp, Artificial Sequence
  Description of Artificial Sequence Synthetic oligonucleotide
ttattattattattattattatttgaaaaggtttgtg

SEQ ID NO: 181 - DNA, 10 bp, Artificial Sequence
  Description of Artificial Sequence Synthetic oligonucleotide
caccagatcc

SEQ ID NO: 182 - DNA, 10 bp, Artificial Sequence
  Description of Artificial Sequence Synthetic oligonucleotide
caccagatct

SEQ ID NO: 183 - DNA, 51 bp, Artificial Sequence
  Description of Artificial Sequence Synthetic oligonucleotide
ccccacaatggatgttaaggttctcaaagggaacagctcggatctggtgtt

SEQ ID NO: 184 - DNA, 42 bp, Artificial Sequence
  Description of Artificial Sequence Synthetic oligonucleotide
gagctgttccctttgagaaccttaacatccattgtggggatg

SEQ ID NO: 185 - DNA, 13 bp, Artificial Sequence
  Description of Artificial Sequence Synthetic oligonucleotide
gtgagaagaaatc

SEQ ID NO: 186 - DNA, 13 bp, Artificial Sequence
  Description of Artificial Sequence Synthetic oligonucleotide
gtgagaagaaatt

SEQ ID NO: 187 - DNA, 51 bp, Artificial Sequence
  Description of Artificial Sequence Synthetic oligonucleotide
agtacagaagatgattgacctggagacaccatccaccccgatttcttctca

SEQ ID NO: 188 - DNA, 42 bp, Artificial Sequence
  Description of Artificial Sequence Synthetic oligonucleotide
ggggtggatggtgtctccaggtcaatcatcttctgtactggg

SEQ ID NO: 189 - DNA, 11 bp, Artificial Sequence
  Description of Artificial Sequence Synthetic oligonucleotide
cagcaaatacc

SEQ ID NO: 190 - DNA, 11 bp, Artificial Sequence
  Description of Artificial Sequence Synthetic oligonucleotide
cagcaaatact

SEQ ID NO: 191 - DNA, 51 bp, Artificial Sequence
  Description of Artificial Sequence Synthetic oligonucleotide
tcaattgttcgaggcttaagagtaaaggagtagatttttcggtatttgctg

SEQ ID NO: 192 - DNA, 36 bp, Artificial Sequence
  Description of Artificial Sequence Synthetic oligonucleotide
gaaaaatctactcctttactcttaagcctcgaacaa

SEQ ID NO: 193 - DNA, 13 bp, Artificial Sequence
  Description of Artificial Sequence Synthetic oligonucleotide
gattttgagtcta

SEQ ID NO: 194 - DNA, 13 bp, Artificial Sequence
  Description of Artificial Sequence Synthetic oligonucleotide
gattttgagtctg

SEQ ID NO: 195 - DNA, 51 bp, Artificial Sequence
  Description of Artificial Sequence Synthetic oligonucleotide
aaacacagatgatggagatgtctgcaggtatgtattcatagactcaaaatc

SEQ ID NO: 196 - DNA, 40 bp, Artificial Sequence
  Description of Artificial Sequence Synthetic oligonucleotide
tgaatacatacccgcagacatctccatcatctgtgtttac

SEQ ID NO: 197 - DNA, 12 bp, Artificial Sequence
  Description of Artificial Sequence Synthetic oligonucleotide
ggacaatacaga

SEQ ID NO: 198 - DNA, 12 bp, Artificial Sequence
  Description of Artificial Sequence Synthetic oligonucleotide
ggacaatacagt

SEQ ID NO: 199 - DNA, 51 bp, Artificial Sequence
  Description of Artificial Sequence Synthetic oligonucleotide
tctatttcttcctcactcagagtcttgaactctattagatctgtattgtcc

SEQ ID NO: 200 - DNA, 38 bp, Artificial Sequence
  Description of Artificial Sequence Synthetic oligonucleotide
tctaatagagttcaagactctgagtgaggaagaaatag

SEQ ID NO: 201 - DNA, 11 bp, Artificial Sequence
  Description of Artificial Sequence Synthetic oligonucleotide
tgaggaagaaa

SEQ ID NO: 202 - DNA, 11 bp, Artificial Sequence
  Description of Artificial Sequence Synthetic oligonucleotide
tgaggaagaag

SEQ ID NO: 203 - DNA, 50 bp, Artificial Sequence
  Description of Artificial Sequence Synthetic oligonucleotide
ctctgcaaggaaatattaaatatatttttcagcactttttctatttcttc

SEQ ID NO: 204 - DNA, 43 bp, Artificial Sequence
  Description of Artificial Sequence Synthetic oligonucleotide
tagaaaaagtgctgaaacatatatttaatatttccttgcagag

SEQ ID NO: 205 - DNA, 12 bp, Artificial Sequence
  Description of Artificial Sequence Synthetic oligonucleotide
gaataaggagta

SEQ ID NO: 206 - DNA, 12 bp, Artificial Sequence
  Description of Artificial Sequence Synthetic oligonucleotide
gaataaggagtg

SEQ ID NO: 207 - DNA, 51 bp, Artificial Sequence
  Description of Artificial Sequence Synthetic oligonucleotide
aactggtgagctggatgacaaatagacaagattgttttactccttattcta

SEQ ID NO: 208 - DNA, 42 bp, Artificial Sequence
  Description of Artificial Sequence Synthetic oligonucleotide
aaacaatcttgtctatttgtcatccagctcaccagttatcaa

SEQ ID NO: 209 - DNA, 15 bp, Artificial Sequence
  Description of Artificial Sequence Synthetic oligonucleotide
ataataataaatgtc

SEQ ID NO: 210 - DNA, 15 bp, Artificial Sequence
  Description of Artificial Sequence Synthetic oligonucleotide
ataataataaatgta

SEQ ID NO: 211 - DNA, 51 bp, Artificial Sequence
  Description of Artificial Sequence Synthetic oligonucleotide
accaatttccaagataaccacaggccatctttaaaagacatttattattat

SEQ ID NO: 212 - DNA, 42 bp, Artificial Sequence
  Description of Artificial Sequence Synthetic oligonucleotide
ttttaaagatggcctgtggttatcttggaaattggtgattta

SEQ ID NO: 213 - DNA, 49 bp, Artificial Sequence
  Description of Artificial Sequence Synthetic oligonucleotide
aatttctgggaaggatcagcctcaggtgccttgtatcttccgtttgacg

SEQ ID NO: 214 - DNA, 50 bp, Artificial Sequence
  Description of Artificial Sequence Synthetic oligonucleotide
aatttcttcattctgatctcctagaagacagcaaataccaaaaaatctac

SEQ ID NO: 215 - DNA, 52 bp, Artificial Sequence
  Description of Artificial Sequence Synthetic oligonucleotide
gtctgcaaggaacaaaatgatttactagtaaacacagatgctggagatgtct

SEQ ID NO: 216 - DNA, 50 bp, Artificial Sequence
  Description of Artificial Sequence Synthetic oligonucleotide
gatctaatagagttcaagactctgagtgagaaagaaatagaaaaagtgct

SEQ ID NO: 217 - DNA, 50 bp, Artificial Sequence
  Description of Artificial Sequence Synthetic oligonucleotide
cacaaaccttttcaaataataataataataataaaaataaatgtctttta

SEQ ID NO: 218 - DNA, 51 bp, Artificial Sequence
  Description of Artificial Sequence Synthetic oligonucleotide
ccccacaatggatgttaaggttctcaaagggaacagctcagatctggtgtt

SEQ ID NO: 219 - DNA, 51 bp, Artificial Sequence
  Description of Artificial Sequence Synthetic oligonucleotide
agtacagaagatgattgacctggagacaccatccaccccaatttcttctca

SEQ ID NO: 220 - DNA, 51 bp, Artificial Sequence
  Description of Artificial Sequence Synthetic oligonucleotide
tcaattgttcgaggcttaagagtaaaggagtagatttttcagtatttgctg

SEQ ID NO: 221 - DNA, 51 bp, Artificial Sequence
  Description of Artificial Sequence Synthetic oligonucleotide
aaacacagatgatggagatgtctgcaggtatgtattcacagactcaaaatc

SEQ ID NO: 222 - DNA, 51 bp, Artificial Sequence
  Description of Artificial Sequence Synthetic oligonucleotide
tctatttcttcctcactcagagtcttgaactctattagaactgtattgtcc

SEQ ID NO: 223 - DNA, 50 bp, Artificial Sequence
  Description of Artificial Sequence Synthetic oligonucleotide
ctctgcaaggaaatattaaatatatttttcagcactttttctacttcttc

SEQ ID NO: 224 - DNA, 51 bp, Artificial Sequence
  Description of Artificial Sequence Synthetic oligonucleotide
aactggtgagctggatgacaaatagacaagattgtttcactccttattcta

SEQ ID NO: 225 - DNA, 51 bp, Artificial Sequence
  Description of Artificial Sequence Synthetic oligonucleotide
accaatttccaagataaccacaggccatctttaaaatacatttattattat

(19) United States
(12) Patent Application Publication
(10) Pub. No.: US 2004/0241714 A1
(43) Pub. Date: Dec. 2, 2004

(54) Methods of assessment of drug metabolizing enzymes

(76) Inventors: Robert A. Branch, Pittsburgh, PA (US); Marjorie Romkes, Export, PA (US)

Correspondence Address: William G. James, Esq., KENYON & KENYON, 1500 K Street, N.W., Suite 700, Washington, DC, 20005, US

(21) Appl. No.: 10/770,538

(22) Filed: Feb. 4, 2004

Related U.S. Application Data
(60) Provisional application No. 60/444,656, filed on Feb. 4, 2003.

(51) Int. Cl. C12Q 1/68; G01N 33/53; C07H 21/04; C12N 9/02

(52) U.S. Cl. 435/6; 435/7.1; 435/69.1; 435/189; 435/320.1; 435/325; 536/23.2

(57) ABSTRACT

The invention provides a method for assessing drug metabolizing enzyme expression levels in whole blood. The invention enables prediction of the effectiveness or safety of a drug therapy by providing a measure of the drug metabolizing capability of the patient. The invention provides a method for detecting and quantifying CYP2D6 mRNA in biological samples, a multiplex assay for detecting SNPs of CYP2D6 gene, and a multiplex assay for detecting SNPs of NAT1 and NAT2.

### FIELD OF THE INVENTION

The present invention relates generally to the field of drug and xenobiotic enzymatic metabolism and specifically to methods of assessing and characterizing such metabolism. In particular, the invention relates to whole blood quantitative tests for assessing a subject's cytochrome P450 (CYP) enzyme activity by assessing CYP gene expression in whole blood. Thus, the present invention provides methods for assessing the activity of the CYP enzymes, especially those involved in drug and xenobiotic metabolism, through assessment of the mRNA expression for the particular CYPs in whole blood. In one embodiment, the invention provides methods for assessing the activity of the CYP2D6 enzyme by measuring CYP2D6 gene expression in whole blood. In other embodiments, the invention provides qualitative tests for assessing and characterizing a subject's metabolic enzyme status through tests for genomic variants in the genes coding for metabolic enzymes. For example, the invention relates to methods for detecting the presence of single nucleotide polymorphisms (SNPs) of the CYP2D6 gene. The invention further relates to a qualitative test for detecting SNPs of two forms of the gene coding for the metabolic enzyme N-acetyltransferase (NAT1 and NAT2) in biological samples. Also disclosed are methods of utilizing the invention to tailor pharmaceutical therapies, predict drug interactions, and diagnose disease conditions through qualitative and quantitative tests for the presence and expression of genes encoding drug metabolizing enzymes.

### BACKGROUND OF THE INVENTION

It is well documented that the rate at which drugs are metabolized varies between individuals and in the same individual depending on, among other things, that individual's health status or concomitant use of other medications. It would, therefore, be useful to be able to identify and measure biomarkers that predict or characterize a particular individual's ability to metabolize a given compound or group of compounds. This information would provide, for example, the ability to tailor drug selection and dosing for optimal drug efficacy and safety. In the same vein, it would also be useful to identify variants of genes encoding drug metabolizing enzymes, particularly those containing SNPs, for use in determining the genotype of individuals, and correlating such genotypic information with clinically significant phenotypical characteristics such as metabolic enzyme activity. Such diagnostic tools for assisting physicians in rational therapeutic decision making regarding the use of drugs metabolized by various enzymes are in great need. Indeed, in November 2003, the FDA published the proposed Guidance for Industry on Pharmacogenomic Data Submissions in which it recommended the submission of valid and probable valid pharmacogenomic biomarkers as part of some IND's, NDA's and BLA's. Notice of Guidance for Industry on Pharmacogenomic Data Submissions, Fed. Reg. Vol. 68, No. 213, pp. 62461-63 (draft guidance available at: http://www.fda.gov/cber/gdlns/pharmdtasub.htm) incorporated herein by reference in its entirety). We have developed various approaches to meeting this need, including: quantitatively assessing in vivo CYP enzyme activity by measuring CYP gene expression in whole blood; qualitative multiplex tests for SNPs of CYP2D6; and qualitative multiplex tests for SNPs of NAT1/NAT2. Information derived from these approaches will help personalize therapeutic decision making.

### A. Drug Metabolism

The desirable and undesirable effects of a drug arise from the concentration of the drug at its site(s) of action. The concentration of a drug at its site(s) of action is generally related to the blood concentration of the drug, which is affected by the amount of drug administered in conjunction with its absorption, distribution, metabolism and excretion. Elimination of a drug or its metabolites occurs either by metabolism, usually by liver or intestinal cells, followed by excretion, or by excretion alone, usually through the kidneys or liver.

Metabolism of drugs (and other xenobiotics) is often complex; many factors can alter hepatic and intestinal drug metabolism, including the presence or absence of certain metabolic enzyme systems or the concomitant administration of other compounds that effect the activity of those systems. The major site of metabolism in the body is the liver. Metabolism by the liver generally occurs in two Phases: First, drugs are functionalized in Phase I pathways within hepatocytic microsomes; then, in Phase II pathways, the parent drug or metabolite created in Phase I is conjugated. Phase I reactions, which result in the elimination of many drugs, are catalyzed primarily by a group of enzymes known as the cytochrome P450 family of enzymes. In hepatocytes, cytochrome P450 enzymes are located in the endoplasmic reticlum. Although the predominant site of cytochrome P450 enzymes is in hepatocytes, the cytochrome P450 enzyme system is also found in intestinal mucosal cells, where it functions to metabolize drugs as they are absorbed from the gastrointestinal tract. These mucosal cell enzyme systems can significantly affect the amount of metabolized/unmetabolized drug that passes into the systemic circulation.

### B. Drug-metabolizing Enzymes

The biotransformation of drugs and other xenobiotics into hydrophilic metabolites is essential for the termination of their biological activity and elimination from the body. Generally, biotransformation reactions generate inactive metabolites that are readily excreted from the body.

As discussed above, drug transformation reactions are classified as either Phase I functionalization reactions or Phase II biotransformation reactions. Phase I reactions introduce or expose a functional group on the parent compound. Phase I reactions generally result in the loss of pharmacological activity, although there are examples of retention or enhancement of activity after Phase I reaction. One example of the clinical exploitation of Phase I reactions is the use of so-called prodrugs. Prodrugs are pharmacologically inactive compounds, designed to maximize the amount of the active species that reaches its site of action. Inactive prodrugs are converted rapidly to biologically active metabolites, often by hydrolysis of an ester or amide linkage. Commonly, prodrugs are converted to their active form via Phase I reactions.

Examples of common Phase I chemical reactions include aromatic hydroxylation, aliphatic hydroxylation, oxidative N-dealkylation, S-oxidation, reduction, and hydrolysis, reactions typically mediated by cytochrome P450s.

1. CYP Activity

The cytochrome P450 enzyme system is one of the most actively studied enzymatic systems and is an important area of study in drug development. CYPs are membrane bound proteins containing a heme moiety with an approximate molecular weight of 450 kD. Most cytochrome P450 enzymes are 400 to 530 amino acids in length. As stated above, CYPs and other mixed function oxygenases are found primarily in the endoplasmic reticulum of hepatocytes, but they are also found in cells of other organs. CYP enzymes are grouped into families and sub-families based on their structural similarity. Generally, families include CYPs with >40% amino acid sequence homology, and are designated by a number, for example CYP2. Subfamilies include CYPs within a family that have >60% amino acid sequence homology. Subfamilies are generally designated by a letter following the number, for example CYP2D. Within the subfamilies, the isoform is indicated by sequential numbers in the order in which they were identified, for example CYP2D6.

Although the CYP isoenzymes generally have similar functional properties, each one is different and has a distinct role. People vary qualitatively and quantitatively with regard to each of the isoenzymes. Thus far, over 30 human CYP 450 enzymes have been identified, out of which six, CYP1A2, CYP2C9, CYP2C19, CYP2D6, CYP2E1, and CYP3A4 appear to be the major drug metabolizing enzymes. Examples of drugs with oxidative metabolism associated with CYP enzymes include acetaminophen, alfentanil, alprazolam, alprenolol, amiodarone, amitriptyline, astemizole, buspirone, caffeine, carbamazepine, chlorpheniramine, cisapride, clomipramine, clozapine, codeine, colchicine, cortisol, cyclophosphamide, cyclosporine, dapsone, desipramine, dextromethorphan, diazepam, diclofenac, diltiazem, encainide, erythromycin, estradiol, felodipine, fluoxetine, fluvastatin, haloperidol, ibuprofen, imipramine, indinavir, indomethacin, indoramin, irbesartan, lidocaine, losartan, macrolide antibiotics, mephenytoin, methadone, metoprolol, mexilitene, midazolam, moclobemide, naproxen, nefazodone, nicardipine, nifedipine, nitrendipine, nortriptyline, olanzapine, omeprazole, ondansetron, oxycodone, paclitaxel, paroxetine, phenacetin, phenytoin, piroxicam, progesterone, propafenone, propranolol, quinidine, ritonavir, saquinavir, sertraline, sildenafil, S-warfarin, tacrine, tamoxifen, tenoxicam, terfenadine, testosterone, theophylline, timolol, tolbutamide, triazolam, verapamil, and vinblastine.

CYPs have been understood for some time to metabolize a large number of drugs and xenobiotics, and recently a number of drugs have been identified as being exclusively or predominantly metabolized by one particular CYP: debrisoquine 4-hydroxylase (CYP2D6), a cytochrome P450 enzyme involved in Phase I metabolism. Drugs metabolized by CYP2D6 vary widely in their clinical use, ranging from antihypertensives to antidepressants and antipsychotics. CYP2D6 is responsible for the metabolism of many psychotherapeutic agents. For each of these drugs, CYP2D6 enzyme activity is a major determinant of drug disposition and therapeutic response. In fact, CYP2D6 (in conjunction with other members of the CYP family) is responsible for metabolizing up to 20% of commonly prescribed drugs. CYP2D6 drug substrates, or chemical entities modified by CYP2D6, include, for example, alprenolol, amitriptyline, chlorpheniramine, clomipramine, codeine, desipramine, dextromethorphan, encainide, fluoxetine, haloperidol, imipramine, indoramin, metoprolol, nortriptyline, ondansetron, oxycodone, paroxetine, propranolol, and propafenone.

There is a wide range of interindividual variability in the pharmacokinetics of all drugs metabolized by CYPs. For example, in the case of CYP2D6 metabolized drugs, individual variations in drug clearance over a 20-200 fold range have been measured. For drugs with narrow therapeutic windows, the difference between high and low rates of metabolism (based on, for example, CYP2D6 enzyme activity) can result in consequences ranging from the recommended doses of the drug having no effect to drug-related toxicity in the absence of dosage modification. Additionally, Phase I enzymatic activity has been correlated with certain human diseases. For example, excessive CYP2D6 activity has been associated with certain malignancies of the bladder, liver, pharynx, stomach, and lungs. Thus, it would be useful to be able to identify patients having increased or decreased activity of any of the CYP enzymes, particularly those related to drug metabolism, and, in particular, CYP2D6.

In the past, the prediction of CYP activity, as exemplified by CYP2D6 activity, has been based on genotyping or monitoring of blood levels of drugs after initiating clinical therapy. Multiple SNPs and other factors that influence regulation of the amount of enzyme make genotyping a limited method of monitoring drug response, and in addition, genotyping is expensive and cumbersome. At present, genotyping analysis can only distinguish poor metabolizers (PMs) (7% of the population) from extensive metabolizers (EMs)(90% of the population) and ultra rapid metabolizers (UMs)(3% of the population). Even among extensive metabolizers, there are wide variations in enzymatic activity, as demonstrated in FIG. 1, which provides broad ranges of levels of excessive metabolizer and poor metabolizer classification for common CYP2D6 substrates. The invention herein provides a method of distinguishing within the groups of metabolizers, including the extensive metabolizer group, which is 90% of the population, allowing for a finer discrimination within the PM, EM and UM groups.

In the case of CYP2D6, the identification of a limited number of biological markers for that particular CYP has not been successful as a proactive strategy in therapeutic drug monitoring. For example, earlier tests included screening for only the more common polymorphisms at positions 1023, 1661, 1707, 1846, 2549 and 4180. These methods failed to provide accurate correlation between the genotypic results obtained and phenotype observed. Monitoring of blood levels of a drug after initiation of therapy is, by definition, not proactive or predictive. Therefore, there is a need for information that is complementary to genotyping to better understand and quantify enzyme function. Clearly, a reliable, proactive and predictive assessment of CYP activity in a patient is needed. In particular, reliable, predictive measures of any of the CYPs involved in drug metabolism is needed, and, in particular, a reliable measurement of CYP2D6 gene expression as a predictor of in vivo CYP2D6 activity is needed prior to or at the earliest stage of therapy for efficient and effective therapeutic decision making when treatment involves one of the numerous drugs metabolized by CYP2D6.

Therefore, a need exists for an effective test to predict in vivo whole body activity of the CYP enzymes in patients, and in particular CYP2D6 activity, that is more rapid and economical, and can be more easily used prior to treatment with drugs metabolized by CYPs.

An object of the present invention is, therefore, to provide methods for assessing whole body CYP activity. The inventors here are believed to be the first to develop a method of assessing whole body activity of a CYP enzyme by measuring CYP gene expression in whole blood. The invention here provides methods of assessing CYP activity by measuring CYP mRNA expression (as transcribed product) in whole blood. “Transcribed product”, “transcribed RNA” and “transcribed cDNA” are used herein to mean nucleic acid that results from reverse transcription of mRNA.

In one aspect, the invention involves measuring mRNA expression for one or more CYP enzymes in whole blood and normalizing that measurement to account for the variability in sample size (i.e., the amount of tissue/number of cells in the sample), and from that measurement determining the degree of whole body activity for the CYP(s) in question. In a preferred embodiment, normalization involves comparison of the measured CYP mRNA expression to the expression of a control gene. “Housekeeping gene”, “reference gene” or “control gene” are used interchangeably herein to mean any constitutively or globally expressed gene whose presence enables normalization or assessment of CYP gene mRNA expression levels. Such normalization is a comparison (or ratio) between the levels of expression of the gene of interest and the determined overall level of constitutive gene transcription in the sample. “Housekeeping genes”, “reference genes” or “control genes” can include, but are not limited to, βactin, βglucuronidase (β-GUS), glyceraldehyde-3-phosphate dehydrogenase (GAPDH), 18S ribosomal RNA (rRNA), β2-microglobulin, acidic ribosomal protein, cyclophilin, phosphoglycerokinase, hypoxanthine ribosyl transferase, transcription factor IID (TATA binding protein).

In a preferred embodiment, the invention provides a test for quantifying CYP2D6 expression from a whole blood sample. More specifically, it is an object of the present invention to provide tests using Q-PCR technology for determining the concentration of mRNA in whole blood for one or more CYP enzymes. As a preferred embodiment, it is an object of the invention to provide Q-PCR tests for determining the concentration of CYP2D6 mRNA in whole blood. In a further preferred embodiment, it is an object of the invention to compare the concentration of CYP2D6 mRNA in whole blood to the expression of the β-Gus gene. It is a further object of the invention to provide methods of tailoring drug therapy and diagnosing diseases by measuring CYP mRNA expression in whole blood. It is also an object of the present invention to provide kits for assessing CYP gene expression, and in particular CYP2D6 gene expression, in a whole blood sample.

In the case of CYP2D6, the quantitation of whole blood CYP2D6 mRNA levels will serve as a diagnostic tool for assisting physicians in rational therapeutic decision-making for drugs metabolized by CYP2D6, and therefore serve several important clinical needs. Whole blood CYP2D6 MRNA levels provide an indication of drug response and genetic variation. This is important information necessary for optimized efficacy and safety of drug therapy. This information will help personalize therapeutic decision-making by guiding the selection of dose at initiation of therapy into either a low, intermediate or high dose schedule depending on whether high, normal or low rates of metabolism are predicted, respectively; or for drug exclusion if a high-risk profile is identified, i.e., by slow metabolism. In addition, this test could be used to assist protocol design for clinical trials of investigational drugs by basing the inclusion or exclusion of potential study participants on the results of the CYP2D6 expression assay. Furthermore, this test provides information to explain inter-subject variation of drug response within a group of patients.

2. Metabolic Enzyme SNPs

Several of the drug metabolizing enzymes are polymorphic, i.e., within a population, more than one nucleotide (G, A, T, C) is found at a specific position in a gene. Polymorphisms may provide functional differences in the genetic sequence through changes in the encoded polypeptide, changes in mRNA stability, or changes in the binding of transcriptional and translational factors to the DNA or RNA. A person with a specific polymorphic variation could therefore have more or less of a specific enzyme, or the expressed enzyme may vary in its level of activity. Thus, polymorphism in the genes coding for drug metabolizing enzymes forms one basis for interindividual differences in the efficacy of drug treatment, side effects of drugs and the toxic and carcinogenic action of xenobiotics. Furthermore, polymorphisms often occur as single nucleotide polymorphisms (SNPs), which are DNA sequence variations that occur when a single nucleotide in the genome sequence varies between different individuals. Thus, it is apparent that measuring CYP gene expression and genotyping subjects to detect, for example, variant CYP2D6 gene alleles that are associated with increased or decreased enzyme activity would be valuable in predicting individual dose requirements for certain drugs as well as avoiding drug over dosage-related side effects and drug interactions in clinical practice.

SNPs are generally biallelic systems, that is, there are two alleles that an individual may have for any particular marker. SNPs that are found approximately every kilobase offer the potential for generating very high density genetic maps, which are extremely useful for developing haplotyping systems for genes or regions of interest, and because of the nature of SNPs, they may in fact be the polymorphisms associated with the disease phenotypes under study. The low mutation rate of SNPs also makes them excellent markers for studying complex genetic traits.

It is known, for example, that polymorphisms in the CYP2D6 gene correlate with enzyme activity measured by phenotyping with dextromethorphan or debrisoquine (Sachse et al. (1997) Am. J. Hum. Genet. 60:248-295). In fact, the CYP2D6 gene is a prototypical example where the initial identification of polymorphic phenotypic distribution of drug oxidation, with characterization of poor metabolizers (PM) and extensive metabolizers (EM) of debrisoquine and spartein were shown to be due to the presence of mutant alleles.

For example, the CYP2D6 poor metabolizer is a phenotype characterized by a monogenic autosomal recessive defect in which little or no CYP2D6 enzyme is detected in vivo. This phenotype is caused by several mutant alleles of the CYP2D6 gene, the most common of which is a *4B, wherein the nucleotide transition at the junction of intron 3 and exon 4 leads to incorrect splicing of the mRNA. Observations of phenotypic variances provided the incentive to identify and sequence the CYP2D6 gene.

In addition to a gene deletion, at least 76 allelic variants have been identified for CYP2D6, many of which are associated with either decreased or enhanced metabolic activity. These allelic variants are mostly made up of one or more SNPs, but also include insertions and deletions. It is clearly of significant clinical importance to identify and characterize the CYP2D6 SNPs because the enzyme is responsible for the predominant or exclusive metabolism of many structurally diverse and often therapeutically important compounds.

Some mutations in the CYP2D6 gene that result in poor metabolizer phenotype include, for example: (i) the complete deletion of the gene resulting in an absence of protein expression, (e.g., CYP2D6*5); (ii) premature termination of the transcript (e.g., CYP2D6*8); and, (iii) a splicing defect at the junction of exons 3 and 4 (e.g., CYP2D6*4). (See Marez, D. et al., Pharmacogenetics 7; 193-202, 1997). Individuals having these mutations, and therefore the poor metabolizer phenotype, are susceptible to toxicity and adverse effects on repeated exposure to compounds metabolized by CYP2D6. For example, poor metabolizers accumulate increased levels of tricyclic antidepressants and are more susceptible to adverse effects, which may be interpreted as symptoms of depression. The intermediate metabolizer phenotype, or CYP2D6*2 variant is associated with SNPs in exon 6 (C>T), exon 3 (G>C) and exon 9 (G>C). These individuals have residual but diminished CYP2D6 enzymatic activity. At the other end of the spectrum of metabolic activity are ultra rapid metabolizers, who require higher dosages of drugs metabolized by CYP2D6 to attain adequate therapeutic effect. These patients may be confused with those that are non-compliant. The genotype responsible for the ultra-rapid metabolizer is duplication or multiplication of the CYP2D6 gene, although other variants are likely because gene duplication is reported to identify only 20% of this group. Adding to the complexity are the findings that SNPs in the 5′-flanking regulatory element regions of CYP2D6 may result in alterations in enzymatic activity.

Thus, in addition to being able to identify and quantify a person's expression of CYP2D6, it is equally important to know whether a patient has one or more CYP2D6 SNPs associated with increased or decreased CYP2D6 activity. To date, the prediction of CYP2D6 activity has been based on genotyping. Current methods for determining the presence or absence of the most common variants of CYP2D6 gene include allele-specific polymerase chain reaction, restriction fragment length polymorphism-PCR (RFLP-PCR), allele-specific hybridization, single-strand conformational polymorphism-PCR (SSCP-PCR) and sequencing. However, these methods are time consuming, labor intensive, and expensive. Furthermore, these methods have not provided a method for screening all possible variant alleles of CYP2D6.

Current genotyping technologies developed to identify SNPs also include high-throughput microarray formats. Matrix Assisted Laser Desorption/Ionization Time-of-Flight (MALDI-TOF) Mass Spectrometry, TaqMan® allele discrimination, and sequencing are technologies that vary in terms of flexibility to add additional SNPs, sensitivity, accuracy of SNP classification, scalability, automation compatibility, ease of assay optimization and efficiency in test turnaround time. And while some high throughput genotyping technologies, including MALDI-TOF Mass Spectrometry, and microarray analysis can provide a multiplex assay for variant alleles, these technologies are very expensive and it may be difficult to add newly-discovered SNPs for screening.

Therefore, there is a need for cost-effective, flexible, reliable and simple multiplex test for determining whether a patient has one or more (CYP2D6) SNPs, to predict in vivo CYP2D6 activity. For CYP2D6, the 76 currently known allelic variants are associated with 60 different polymorphic regions. The number of SNPs within a particular variant allele range from a single SNP (e.g, CYP2D6*1B) to 8 SNPs (e.g., CYP2D6*4G).

It is a further object of the present invention to provide a qualitative multiplex test for SNPs of CYP2D6 in a patient using microsphere-based assays and flow cytometry, wherein the multiplex sequence determination allows for a flexible, reliable, and cost-effective method for detecting SNPs in DNA.

Like the CYP polymorphisms, the acetylation polymorphism is one of the most common genetic variations in the transformation of drugs and xenobiotics, including, for example, the activation of arylamines in tobacco smoke. Drugs metabolized by the acetylation pathway include isoniazid, sulfamethazine and other sulfonamides, procainamide, hydralazine, dapsone and caffeine. The xenobiotic-metabolizing enzyme that is responsible for acetylation is human N-acetyltransferase, which exists in two forms that can be distinguished by their high specificity for the substrates p-aminobenzoic acid (PABA, for NAT 1) and isoniazid (INH, for NAT2).

Genetic polymorphisms in the NAT2 gene are among the earliest discovered in metabolic enzymes. The NAT2 gene locus is the site of the classical isoniazid acetylation polymorphism. Deficiency in NAT2 is responsible for toxicity associated with several drugs. The variances in the ability to metabolize these compounds distinguish phenotypically slow and fast acetylators of INH drugs. This variability in metabolizing capacity is due to the presence of mutants alleles found in the DNA sequence which differ from the wild-type allele. These mutant alleles are important in genotyping of polymorphic alleles to identify an individual's drug metabolism phenotype. More than 50% of Caucasians are homozygous for a recessive trait, resulting in deficiencies in NAT2 and the slow acetylator phenotype (Blum, et al., Proc. Natl. Acad. Sci 1991;88: 5237-5241). Several mutant NAT2 alleles have been found in Caucasians and Asian individuals (Blum et al., 1991). Specifically, for NA T2, two mutant alleles M1 (NAT2*5A) and M2 (NAT2*6A) account for over 90% of the alleles associated with slow acetylation in Caucasians. (Blum, et al., 1991). The NAT2*7A allelic variant is relatively rare among Caucasians and African Americans, occurring in only 2% of the alleles associated with slow acetylation in the two populations. The NAT2*14A allele appears to be of African origin, occurring in 9% of African Americans, and no Caucasians (Bell, et al., Carcinogenesis 1993; 14: 1689-1692). Thirteen point mutations have been reported in NAT2*, each a single base-pair substitution, resulting in 28 known allelic variants. (Http://www.louisville.edu/medschool/pharmacology/NAT.html.). Similarly, a number of single nucleotide substitutions and deletions have been identified for NAT1*, resulting in 26 allelic variants. NAT] metabolizes substrates whose disposition is unrelated to the isoniazid acetylator phenotype, but it also displays significant genetic variation in human populations (Grant et al., Pharmacogenetics, 3:45-50).

Even though knowledge of the existence of NAT1 and NAT2 variants has been available for some time, there is still a need for improved high throughput genotyping of common mutant alleles of NAT1 and NAT2 to assist in their identification. Detection of SNPs in the NAT1 and NAT2 genes provides important information that can be applied to genotyping applications, including collecting genotyping information for correlating with other test information. For example, the detection of SNPs in drug metabolism genes provides information on patient drug response. Drugs that undergo acetylation include isoniazid, amonafide, caffeine, hydralazine, procainamide, sulfapyradine, dapsone, nitrazepam, and sulfasalazine. Thus, there is a need for a effective method of detecting SNPs of NAT1 and NAT2 that is rapid and cost-effective.

Accordingly, it is an object of the present invention to provide a rapid, low cost, flexible and reliable, qualitative multiplex test for SNPs of NAT1 and NA T2. We have developed such a qualitative test for SNPs of NAT1 and NA T2. In a preferred embodiment, this test uses a multiplex microchip technology for simultaneously screening for SNPs of NAT1 and NAT2 using an active electronic microchip array. (For discussion of NAT1 SNPs see, e.g, Deitz A C, et al., Anal Biochem 1997; 253: 219-224; Bell, D A, et al., Cancer Res 1995; 55: 5226-5229; and Doll M A, et al., Biochem Biophys Res Commun 1997; 233: 584-591; and for a review of NAT2 genotyping, see, e.g., Blum M., et al., Proc. Natl. Acad. Sci 1991; 88: 5237-5241; Bell D., et al., Carcinogenesis 1993; 14: 1689-1692; Sohni YR, et al., Clin Chem 2001; 47: 1922-1924; and Behrensdorf H A, et al., Nucleic Acids Res 2002; 30(14) 64; for a general discussion of location of NAT genes, see Hickman, et al., 1994, Biochem J. 297:441-445, the disclosures of which are incorporated herein by reference.)

### SUMMARY OF THE INVENTION

The present invention provides quantitative methods for measuring the expression of a CYP enzyme in a subject that comprise measuring the expression of the CYP enzyme gene in a biological sample. The CYP enzymes that may be assessed according to the invention include CYP1A1, CYP1A2, CYP1B1, CYP2C8, CYP2C9, CYP2C18, CYP2C19, CYP2D6, CYP2E1, CYP3A4, and CYP3A5. In addition, the invention provides methods for measuring the activity of a CYP enzyme in a subject that comprise measuring the expression of the CYP enzyme gene in a biological sample. Preferably, the sample is a whole blood sample. In a preferred embodiment the invention comprises measuring the expression of the CYP enzyme gene by measuring the expression of mRNA for the CYP enzyme. Moreover, the invention may include normalizing the measured expression of the CYP enzyme gene. In particular, such normalization may include comparing the measured expression of the CYP enzyme gene to the expression of a control gene. In a preferred embodiment, the control gene is β-GUS although other control genes may be used as described herein.

The inventive methods described here for measuring CYP enzyme expression in a sample comprise isolating and reverse transcribing RNA from the sample to obtain a transcribed product (e.g., cDNA), subjecting the transcribed product to amplification to obtain an amplified product, and determining the amount of transcribed product. In a preferred embodiment, the methods further include comparing the determined amount of transcribed product to the determined amount of transcribed product for a control gene. Preferably, the amplification step is carried out using PCR and the amount of amplified product is determined using TAQMAN® analysis, although other means of measuring the amount of amplified product are described herein. In preferred embodiments, the methods of the invention may be applied to CYP1A1, CYP1A2, CYP1B1, CYP2C8, CYP2C9, CYP2C18, CYP2C19, CYP2D6, CYP2E1, CYP3A4, and CYP3A5. The primers suitable f amplifying the transcribed products for each of these enzymes are listed in Tables 1 and 2.

In a particularly preferred embodiment, the present invention provides quantitative methods for measuring the expression of CYP2D6 enzyme in a subject that comprise measuring the expression of the CYP2D6 enzyme gene in a biological sample. In a more preferred embodiment, the present invention provides a quantitative test for CYP2D6 mRNA in a biological sample. This test comprises the steps of: isolating RNA from the sample; and determining the quantity of CYP2D6 mRNA relative to the quantity of a control gene's MRNA.

In a more particular embodiment, the present invention provides a test for detecting the presence or quantity of expression of the CYP2D6 gene in a blood sample comprising: isolating RNA from the blood sample, reverse transcribing the RNA to cDNA, subjecting the cDNA to amplification using a pair of oligonucleotide primers that hybridize to a region of the CYP2D6 gene to obtain an amplified sample, comparing the amount of CYP2D6 mRNA to an amount of mRNA of a control gene, and determining the amount of CYP2D6 expression. The presence or amount of CYP2D6 mRNA indicates expression of a cellular CYP2D6 gene, and correlates with CYP2D6 activity in the whole body. The endogenous control gene may be, for example, β-glucuronidase.

In another embodiment of this aspect of the invention, oligonucleotide primers are provided having the sequence of CYP2D6. Ftaq (SEQ ID NO: 1) or CYP2D6. Rtaq (SEQ ID NO:2), and sequences substantially identical thereto. The invention also includes oligonucleotide primers having a nucleotide sequence that hybridizes to SEQ ID NO:4 or SEQ ID NO:5, or their complements under stringent conditions, for example as described herein. In a further embodiment, the amplicons are hybridized with probes attached to fluorescently-tagged microspheres, which allow detection of SNPs in CYP2D6 genes in the sample by flow cytometry, for example as described further herein. In addition, the invention includes primers having the nucleotide sequences of (or sequences that hybridize to) SEQ ID NOS. 69, 70, 72, 73, 75, 76, 78, 79, 81, 82, 84, 85, 87, 88, 90, 91, 93, and 94, for amplifying cDNA transcribed from mRNA encoded by the genes for CYP1A1, CYP1A2, CYP1B1, CYP2C8, CYP2C9, CYP2C18, CYP2C19, CYP2E1, and CYP3A4, respectively.

In yet another aspect, the invention provides methods for determining a patient's therapeutic regimen for a drug metabolized by a CYP enzyme that comprises obtaining a biological sample from the patient, isolating and reverse transcribing RNA from the sample to obtain a transcribed product, subjecting the transcribed product to amplification, determining the amount of CYP amplified product, comparing (or creating a ratio of) the amount of CYP amplified product and the amount of amplified product for a control gene, and selecting a therapeutic regimen based on the comparison (or ratio). In a preferred embodiment the comparison or ratio is compared to those for subjects in normal or control populations, or in other particular populations in which the level of CYP activity has been correlated to such comparisons or ratios. In a preferred aspect of the invention there is provided a method for determining a therapeutic regimen for a patient, comprising isolating RNA from a whole blood sample; determining gene expression level of CYP2D6 in the sample; comparing the CYP2D6 gene expression levels in the sample with predetermined levels for expression of the CYP2D6 gene; and determining a therapeutic regimen based on results of the comparison of the CYP2D6 gene expression level with the predetermined levels. In a further embodiment, the invention provides a method for selecting a drug therapy based on the magnitude of CYP2D6 gene expression. In yet a further embodiment, the invention provides a method for selecting a therapeutic dosage of a particular pharmaceutical or selecting a dosing regime.

In yet another aspect, the invention provides a method comprising correlation of results of CYP2D6 quantitation with results of prior art test(s) including in vivo measures of CYP2D6 enzyme activity. These measures include, for example, debrisoquine 4-hydroxylase activity and the sparteine metabolic rate, and D-demethylation of R and S venlafine.

In another aspect, the invention provides a qualitative test for SNPs of the CYP2D6 gene in a sample. This embodiment of the present invention comprises: isolating nucleic acid from a sample, amplifying the sample by PCR, hybridizing the amplicons with tagged probes, and detecting the presence of the SNPs in the CYP2D6 genes, for example as described herein. In yet another embodiment, the present invention provides methods for determining a patient's therapeutic regimen for a drug metabolized by CYP2D6 that comprises selecting the patient's therapeutic regimen based on the presence or absence of CYP2D6 SNPs.

In another embodiment, the present invention provides a multiplex test for detecting previously identified SNPs of the CYP2D6 gene using microspheres to detect multiple homogeneous and heterogeneous SNPs in one reaction.

In another aspect, the invention provides a multiplex qualitative diagnostic assay for detecting previously identified SNPs in the NAT1 and NAT2 genes using a microchip array detection format for detecting SNPs in one reaction.

In another aspect of the invention there is provided a method for determining a therapeutic dosage regimen for a patient comprising isolating DNA from a biological sample; determining presence of SNPs of NAT1 and NAT2 genes in the sample; and determining a therapeutic regimen based on results of the presence or absence of SNPs of NAT1 and NAT2 genes.

### BRIEF DESCRIPTION OF THE FIGURES

FIG. 1 graphs the distribution of CYP2D6 enzyme activity measured by the conversion of Debrisoquine to 4 hydroxydebrisoquine (the DBRR test) for certain CYP2D6*4 variant genotypes. In vivo CYP2D6 enzyme activity was evaluated in human subjects genotyped as homozygous wildtype (H), heterozygotes (HM) and homozygous variant *4 (PM) individuals. FIG. 2 correlates the level of bufurolol metabolism with CYP2D6 mRNA concentrations in human liver samples.

FIG. 3 illustrates debrisoquine recovery ratio (DBRR) compared to concentrations of MRNA for CYP2D6 in PBMCs in 78 healthy volunteers (rₛ=0.56, p<0.001).

FIG. 4 illustrates quantitation of CYP2D6 MRNA using TaqMan® realtime PCR. The curves (18 and 20) represent two different PCR reactions with a delta Cₜ of 2 cycles. This represents a four-fold difference in starting template.

FIG. 5 illustrates a CYP2D6 multiplex SNP detection. Samples 4 and 6 are classified as heterozygous C2850T and the remaining samples are homozygous wildtype for all three SNPs. Microsphere SNP results were confirmed by sequencing analysis.

FIG. 6 illustrates realtime PCR results of CYP2D6 amplification plot.

FIG. 7 illustrates realtime PCR results of CYP2D6 quatitation for nine samples.

FIG. 8 illustrates data generated for each microsphere in tabular form as mean fluorescence intensity units.

FIG. 9 illustrates data generated for each microsphere in graphic form as mean fluorescence intensity units.

FIG. 10 illustrates single exon PCR products for the Luminex based CYP2D6 analysis on a polyacrylaminde gel.

FIG. 11 illustrates results for multiplexed PCR for exons 2, 6 and 8 CYP2D6 analysis.

FIG. 12 illustrates the location of CYP2D6 SNP alleles.

FIG. 13 illustrates examples of primers and probes sequences for use to detect specific NAT1 SNPs.

FIG. 14 illustrates examples of primers and probes sequences for use to detect specific NAT2 SNPs.

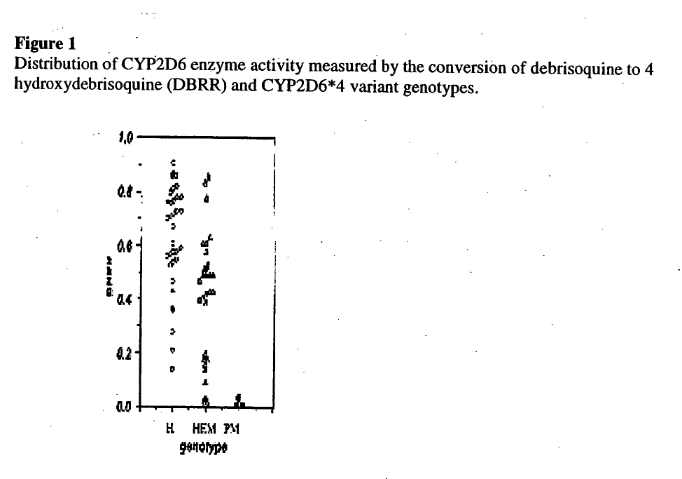

### DETAILED DESCRIPTION OF THE INVENTION

Various aspects of the invention are described in detail in the following subsections.

### A. Whole Body CYP Activity and Whole Blood CYP mRNA Expression

The inventors here have discovered that the expression of a CYP enzyme gene in whole blood is a surrogate for the whole body expression of that CYP enzyme, and, therefore, the whole body activity of the CYP enzyme in a subject. Thus, according to the present invention, the measurement of gene expression for a CYP enzyme gene in a sample may be correlated to the whole body activity of that CYP enzyme. The elements common to the regulation of the activity of CYPs that can be measured in order to predict CYP activity in a patient are best described in relation to a preferred embodiment: CYP2D6. However, this aspect of the invention, i.e., measuring whole body CYP activity through the measurement of CYP MRNA expression in whole blood, applies to other CYPs as well, including, but not limited to, CYP1A1, CYP1A2, CYP1B1, CYP2C8, CYP2C9, CYP2C18, CYP2C19, CYP2E1, CYP3A4 and CYP3A5. (See Table 2 for CYP primer and probe sequences). Like all CYPs, the regulation of CYP2D6 expression is in part dependent on the synthesis of mRNA for CYP2D6. Previously, investigators have shown that circulating peripheral blood mononuclear cells (PBMC's), which include lymphocytes and monocytes, express some forms of cytochrome P450 mRNA (See Cosma et al., 1993, Landi et al., Omiecinski et al., 1900, Redlich et al., 1989, Vanden-Heuvel et al., 1993). We developed methods to evaluate levels of CYP2D6 mRNA in liver samples or PBMC's (FIG.s 2 and 3), including the reverse transcription-polymerase chain reaction-fluorescein antifluorescein enzyme linked oligonucelotide sorbent assay (RT-PCRIFAF-ELOSA) that correlates concentration of CYP2D6 mRNA from liver tissue or PBMCs obtained from subjects with bufurolol metabolism for in vitro studies and debrisoquine recovery ratio (DBRR) results for in vivo studies, respectively. In subjects, DBRR, a ratio test that quantitates the metabolism of debrisoquine to 4-hydroxydebrisoquine, is used to evaluate the whole body level of CYP2D6 activity as a quantitative biomarker of activity. (Kaisary, A., et al. 1987).

We have shown a significant association between DBRR and the concentration of MRNA from bladder tissue for CYP2D6 in bladder mucosal tissue, suggesting coordinated whole body regulation of this enzyme and regulation of mRNA levels in that tissue. More recently, this observation has been extended to in vitro tests using human liver tissue where we have used the hydroxylation of bufurolol in liver microsomal preparation as a biomarker of CYP2D6 activity. In this instance, of the 20 livers evaluated, the range of interindividual expression for both MRNA level for CYP2D6 and in vitro enzyme activity were extremely wide, yet there was a significant association (rₛ=0.85 P<0.001) between the variables (see FIG. 2.)

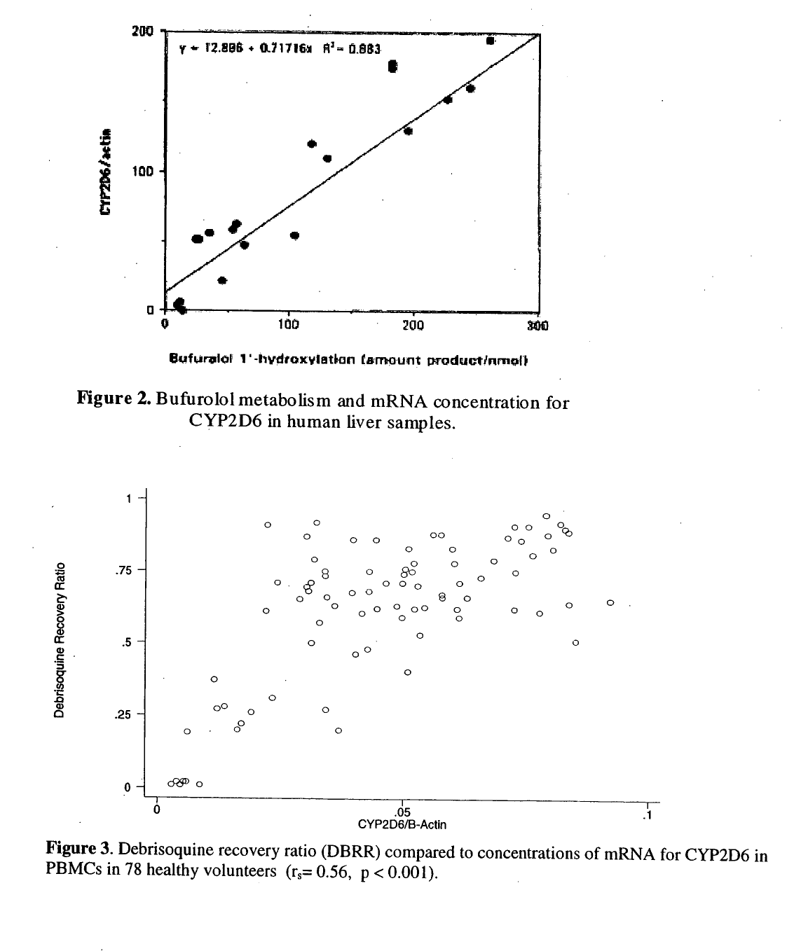

This association suggested that when active CYP2D6 was expressed, regulation of mRNA for CYP2D6 was a major determinant of this activity. Further, this observation implied that there was little interindividual variation in post translation modification or rate of enzyme degradation. Similarly, when we extended this study to include healthy volunteers, we observed a wide variation of both DBRR and concentrations of mRNA for CYP2D6 in PBMCs (see FIG. 3). When concentrations of mRNA for CYP2D6 in blood were compared to DBRR, there was a significant correlation best explained by a nonlinear relationship (rₛ=0.56 P<0.001, FIG. 3). These relationships strongly suggest that CYP2D6 regulation is coordinated between different sites that contribute to in vivo enzyme activity. However, both the liver tissue and the PBMC tests used required labor intensive and costly processing to extract and quantitate mRNA.

The quantitative test for CYP2D6 expression we have developed comprises the steps of: isolating total RNA from the whole blood sample; reverse transcribing the RNA to cDNA, subjecting the cDNA to amplification using a pair of oligonucleotide primers that hybridize to a region of the CYP2D6 gene to obtain an amplified product; comparing the amount of CYP2D6 amplified product to an amount of amplified product for a control gene; and determining the amount of CYP2D6 expression. We have developed a primer pair and a labeled probe to a target sequence of CYP2D6 to perform this test assay.

While CYP2D6 is the preferred embodiment of the invention, as mentioned above, the invention is also applicable to other CYP enzymes. Thus, the present invention includes isolating RNA for a CYP enzyme from whole blood, reverse transcribing the niRNA to cDNA (the transcribed product), amplifying the CYP cDNA using oligonucleotide primers, normalizing the measured CYP amplified product to account for differences in cell number in sample size, and determining the amount of CYP expression. Normalization is necessary to account for the variability of measured CYP mRNA expression caused by differences in sample size. In general, normalization reduces the CYP mRNA measurement to approximate that found on a per cell basis. Measuring the amount of mRNA expressed in a single cell would be a method of normalizing the measurement, but other methods would be apparent to persons of skill in the art. In a preferred embodiment, the CYP mRNA measurement is normalized by comparing the mRNA expression to the expression of a control gene.

As used herein, a “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced. For use in this diagnostic test, preferably the oligonucleotide primer contains at least about 15-25 nucleotides. “Probe” is defined as an oligonucleotide capable of binding to a target nucleic acid of complementary sequence through one or more types of chemical bonds, usually through complementary base pairing by hydrogen bond formation. As used herein, an oligonucleotide probe may include natural (i.e. A, G, C or T) or modified bases (e.g., 7-deazaguanosine and inosine). Additionally, an oligonucleotide probe may bind to amplicons. “Amplicons” are the products of the amplification of nucleic acids by PCR or otherwise. In addition, the bases in oligonucleotide probe may be joined by a linkage other than a phosphodiester bond, so long as it does not interfere with hybridization. As used herein, the term “gene” is intended to refer to the genomic region encompassing the 5′ UTR specifically referred to, e.g., exon 2, intron 5, etc. Combinations of such segments that provide for a complete protein may be referred to generically as a protein coding sequence.

The terms “subject”, “patient” and “individual” are used interchangeably herein to refer to any type of organism from which whole blood samples can be obtained, including humans.

As used herein, “substantially identical” in the nucleic acid context refers to a sequence (or sequences) that will hybridize to a target under stringent conditions, and also means that the nucleic acid segments, or their complementary strands, when compared, are the same when properly aligned, with the appropriate nucleotide insertions and deletions, in at least about 70% of the nucleotides, typically, at least about 80% of the nucleotides, more typically, at least about 90% of the nucleotides, usually, and at least about 95-98% of the nucleotides most typically. Selective hybridization exists when the hybridization is more selective than total lack of specificity. See, Kanehisa, Nucleic Acids Res., 12:203-213 (1984).

“Control” populations and “healthy” populations are used herein to refer to representative populations of subjects, in terms of their CYP enzyme activity. (For an example, see Carcillo J A, et al. Coordinated intrahepatic and extrahepatic regulation of cytochrome P4502D6 in healthy subjects and in patients after liver transplantation Clin Pharn and Ther 2003; 73 (5):456-467, which is incorporated herein by reference in its entirety). These terms are generally referred to herein in the context of populations in which normalized whole blood CYP mRNA expression has been measured.

### B. CYP2D6 MRNA EXPRESSION

In a preferred embodiment, the present invention provides a method of quantifying the amount of CYP2D6 MRNA expression in a biological sample relative to expression of a control gene.

To facilitate the quantification of CYP2D6 MRNA, we have developed a quantitative test that uses Q-PCR (or TaqMan®) technology to amplify, detect, and quantify CYP2D6 mRNA in a biological sample. Using this test, a patient's CYP2D6 expression can be assessed by comparing it to a predetermined threshold expression level.

The biological sample to be tested may be collected in any sterile container. Preferably, the sample is whole blood collected by venapuncture. Although the present method can be applied to any type of sample from a patient, samples other than whole blood would require greater difficulty in acquisition and sample processing. For example, procurement of liver samples requires an invasive procedure followed by an extensive processing procedure. To increase the sensitivity and efficiency of the sample collection, it is preferable to use whole blood. Critical to maximum yield of total RNA from the whole blood sample is the storage process following collection. If the whole blood sample is collected in a potassium EDTA tube, the sample must be frozen at −80° C. within 15-30 minutes of collection. Alternatively, an equal volume of Applied Biosystems nucleic acid lysis solution can be added and the sample must be frozen at −80° C. within four hours. If Qiagen PreAnalytiX® tubes are used, samples are processed immediately by centrifugation and separation steps and the samples can be stored at either room temperature for five days, at 4° C. for two weeks, at −20° C. for 12 weeks, or −80° C. for six months before the RNA isolation procedure.

RNA of the individual sample is isolated by the methods known in the art. A number of techniques exist for the purification of RNA from biological samples. Most known reliable techniques for isolating RNA typically utilize either guanidine salts or phenol extraction, as described for example in Sambrook, J. et al., (1989) at pp. 7.3-7.24, and Ausubel, F. M. et al., (1994) at pp. 4.03-4.4.7. The most reliable technique is described in Godfrey T E, et al., J. Mol. Diagnostics. 2000; 2: 84-91. Preferably, RNA is extracted using the guanidinium thiocyanate/phenol/chloroform single step extraction method using Strategene's RNA isolation kit or the Gentra Systems PureScript® kit.

The invention provides primers for the amplification of a nucleic acid sequence of the CYP2D6 gene in the sample using PCR. PCR is a technique well known to those of skill in the art, which is used to amplify small quantities of DNA or RNA in a sample. (See U.S. Pat. Nos. 4,683,195 and 4,683,202 for a description of the PCR method). The primers bind to a specific site on the CYP2D6 cDNA and allow amplification of that particular nucleic acid sequence. Preferably these primers comprise a sequence selected from CYP2D6.Ftaq (SEQ ID NO:1) and CYP2D6.Rtaq (SEQ ID NO:2) or sequences substantially identical thereto. The invention further provides primers for selective amplification of nucleic acid sequences having a sequence that hybridizes to SEQ ID NO:1 or SEQ ID NO:2 or their complements under stringent conditions.

Most preferably, these primers are used together with RNA extracted from a biological sample and reverse transcribed. Table 1 provides the PCR primers for amplification of CYP2D6. The CYP2D6 TaqMan® forward primer (SEQ ID NO:1) recognizes the exons 3 and 4 junctions in order to only amplify cDNA and not genomic DNA. Using Genbank M33388 as the CYP2D6 reference sequence, the F primer is designed to recognize bases 3371-77, 3466-3474. The reverse primer (SEQ ID NO:2) recognizes 3499-3518 and the probe (SEQ ID NO:3) recognizes 3476-3497. This region was chosen in order to avoid amplification of the highly homologous pseudogenes CYP2D7 and CYP2D8.

The primers for β-GUS, which is used as a control gene in a preferred embodiment include oligonucleotide primers having the sequence of GUS.Ftaq (SEQ ID NO:4), GUS.Rtaq (SEQ ID NO: 5) and β-GUS.taqprobe (SEQ ID NO:6) and sequences substantially identical thereto. The TaqMan® probe and assay conditions used for the β-GUS amplicon were based on those developed by Godfrey, T. E., et al., J. Mol. Diagnostics, 200, 2: 84-91, which is incorporated by reference in its entirety.

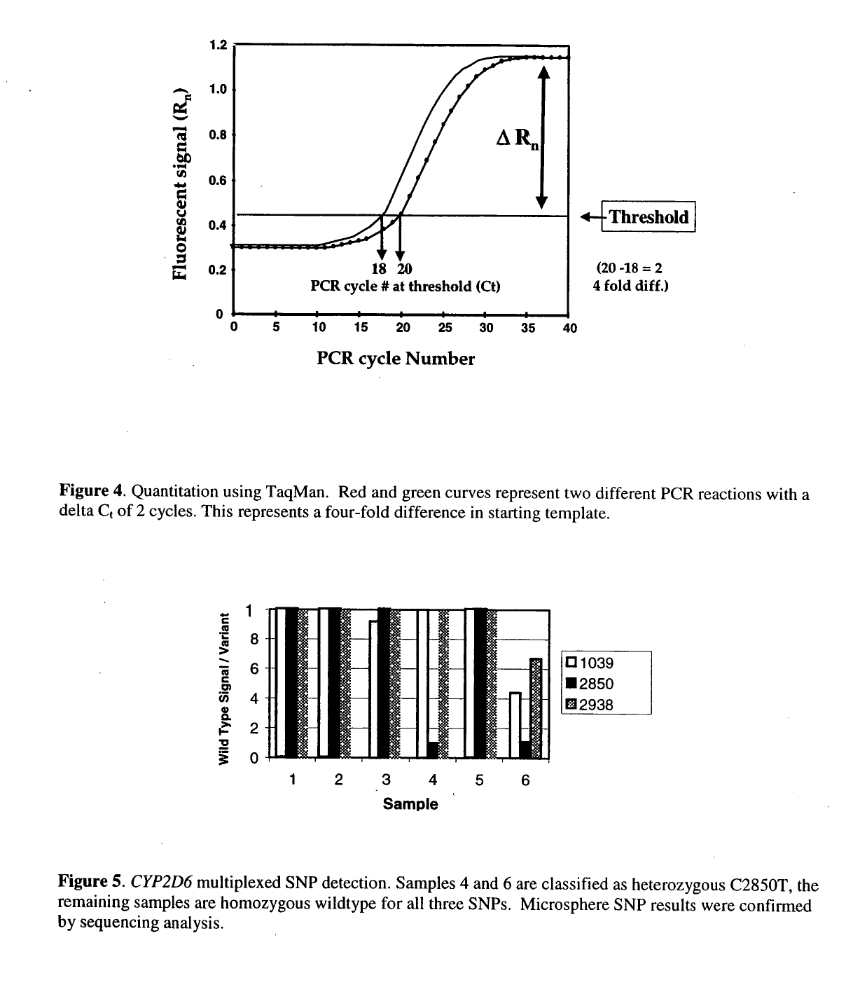

A quantitative PCR system such as the Perkin Elmer Applied Biosystems GeneAmp 7700™ Sequence Detection system (TaqMan® system) was chosen for the ability to continuously measure PCR product accumulation using a dual-labeled fluorogenic oligonucleotide probe, called a TaqMan® probe. TaqMane can be used to quantitate gene expression and provides precise quantification of initial target in each PCR reaction. The amplification plot is examined at a point during the early log phase of product accumulation. This is accomplished by assigning a fluorescence threshold above background and determining the time point at which each sample's amplification plot reaches the threshold (defined as the threshold cycle number or CT) (FIG. 4). Differences in threshold cycle number are used to quantify the relative amount of PCR target contained within each tube.

Most TaqMan® gene expression analysis studies utilize a relative expression calculation similar to standard assays such as used in our RT-PCR/FAF-ELOSA. Expression of the gene of interest is reported relative to expression of an endogenous control gene, which is assumed to have equal expression in all tissues in the study. In this way, expression levels can be compared from tissue to tissue. However, in the present invention, the relative expression of MRNA species has been calculated using a comparative CT method (FIG. 4). In addition, the present invention provides a method of reporting expression of CYP2D6 relative to the endogenous control gene β-glucuronidase (β-GUS). During the validation of this TaqMan® assay, PCR efficiency was required to be >95% when the amplicon was designed with strict parameter limitations, i.e., 95% efficiency of the PCR reaction. This method of relative quantitation requires that the PCR efficiency of the CYP2D6 and β-GUS amplicons be very similar. Our β-GUS amplicon has a PCR efficiency of approximately 98% and the CYP2D6 amplicon efficiency is 99%.

In this embodiment, the invention involves first determining the quantity of CYP2D6 mRNA in the sample by using a pair of oligonucleotide primers, preferably primer pair CYP2D6.Ftaq (SEQ ID NO:1) and CYP2D6.Rtaq (SEQ ID NO:2), or oligonucleotides substantially identical thereto, for carrying out quantitative polymerase chain reaction. RNA is extracted from the sample by any of the known methods for RNA isolation, as, for example, further described herein.

TABLE 1
PCR primer and probe sequences

| | |
|---|---|
| PRIMER or PROBE | OLIGONUCLEOTIDE SEQUENCE |

| | |
|---|---|
| CYP2D6.Ftaq (SEQ ID NO:1) | 5′-CACTCCGGACGCCCCT-3′ |
| CYP2D6.Rtaq (SEQ ID NO:2) | 5′-GATGACGTTGCTCACGGCTT-3′ |
| CYP2D6.taqprobe (SEQ ID NO:3) | 5′(6FAM)TCGCCCCAACGGTCTCTTGGAC(TAMRA)-3′ |
| GUS.Ftaq (SEQ ID NO:4) | 5′-CTCATTTGGAATTTTGCCGATT-3′ |
| GUS.Rtaq (SEQ ID NO:5) | 5′-CCGAGTGAAGATCCCCTTTTTA-3′ |
| β-GUS.taqprobe (SEQ ID NO:6) | 5′(6FAM)TGAACAGTCACCGACGAGAGTGCTGG(TAMRA)-3′ |

In a preferred embodiment, the invention provides primers allowing quantitative PCR amplification of CYP2D6 mRNA extracted from whole blood samples and reverse transcribed. The method involves a pair of PCR primers that span an intron/exon junction in order to amplify only cDNA. Further, the design of the primer pair was verified by running a nucleotide BLAST search at http://www.ncbi.nlm.nih.gov.BLAST/. Primer sequences were aligned with DNA sequences entered in the databases (using BLAST programs), to confirm homology, and checked for similarities with repetitive sequences or with other loci, elsewhere in the genome.

This embodiment further involves the use of a control or housekeeping gene. Preferably, the control gene is βglucuronidase. However, other control genes that have mRNA levels that are consistent between cell line and tissue types can be used, for example, β-actin, glyceraldehyde-3-phosphate dehydrogenase (GAPDH), 18S ribosomal RNA (rRNA), β2-microglobulin, acidic ribosomal protein, cyclophilin, phosphoglycerokinase, hypoxanthine ribosyl transferase, transcription factor IID (TATA binding protein).

TABLE 2

| | | | |
|---|---|---|---|
| Gene | Forward primer | Reverse primer | Probe |

| | | | |
|---|---|---|---|
| CYP1A1 | 5′- | 5′- | 5′-(FAM)-TGGCAGGTCACGGCGGCC-(TAMRA)-3′ |
| | AGAAAGATCCAAGAG | TGGGATCTGTCAGAGA | (SEQ ID NO:71) |
| | GAGCTAGACAC-3′ | GCCG-3′ | |
| | (SEQ ID NO:69) | (SEQ ID NO:70) | |
| CYP1A2 | 5′- | 5′- | 5′-(FAM)-ACGTCATTGGTGCCATGTGC-(TAMRA)-3′ |
| | ATCGCCTCTGACCCAG | CTCATCGCTACTCTCA | (SEQ ID NO:74) |
| | CTTC-3′ | GGG-3′ | |
| | (SEQ ID NO:72) | (SEQ ID NO:73) | |
| CYP1B1 | 5′- | 5′- | 5′-(FAM)-CGAGTGCAGGCAGAATTGGATCAGG-(TAMRA)-3′ |
| | ACCAGGTATCCTGATG | ACGGTCCCTCCCCACG-3′ | (SEQ ID NO:77) |
| | TGCAGAC-3′ | (SEQ ID NO:76) | |
| | (SEQ ID NO:75) | | |
| CYP2C8 | 5′- | 5′- | 5′-(FAM)-CAACCATAATGGCATTACTGACTTCCGTGCTA- |
| | ACTACCTCATCCCCAA | TTGGATTAGGAAATTC | (TAMRA)-3′ |
| | GGGC-3′ | TTTGTCATCA-3′ | (SEQ ID NO:80) |
| | (SEQ ID NO:78) | (SEQ ID NO:79) | |
| CYP2C9 | 5′- | 5′- | 5′-(FAM)-TGTGGGAGAAGCCCTGGCCCG-(TAMRA)-3′ |
| | TTTCTCAGCAGGAAAA | GGTCAGGAATAAAAA | (SEQ ID NO:83) |
| | CGGATT-3′ | CAGCTCCAT-3′ | |
| | (SEQ ID NO:81) | (SEQ ID NO:82) | |
| CYP2C18 | 5′- | 5′- | 5′-(FAM)-AGGGCCTGGCCCGCATGG-(TAMRA)-3′ |
| | TCAGCAGGAAAACGG | TGGTGGTCAGGAATAA | (SEQ ID NO:86) |
| | ATGTGT-3′ | AAACAGC-3′ | |
| | (SEQ ID NO:84) | (SEQ ID NO:85) | |
| CYP2C19 | 5′- | 5′- | 5′-(FAM)-AAACCAACAGTCTGAATTC-(TAMRA)′-3′ |
| | CTGATCAAAATGGAG | GCTGCAGTGATTACCA | (SEQ ID NO:89) |
| | AAGGAA-3′ | AGT-3′ | |
| | (SEQ ID NO:87) | (SEQ ID NO:88) | |
| CYP2E1 | 5′- | 5′- | 5′-(FAM)-CCTCCCCGCGTTCCATGCG-(TAMRA)-3′ |
| | TTCTCGGGCAGAGGCG-3′ | AAAATGATTCCCCTGT | (SEQ ID NO:92) |
| | (SEQ ID NO:90) | CCCTG-3′ | |
| | | (SEQ ID NO:91) | |
| CYP3A4 | 5′- | 5′- | 5′-(FAM)-TCACAGATCCTGACATGATCAAAACAGTGCT- |
| | TGTGGGGCTTTTATGA | GGTTTGTGAAGACAGA | (TAMRA)-3′ |
| | TGGTC-3′ | ATAACATTCTTT-3′ | (SEQ ID NO:95) |
| | (SEQ ID NO:93) | (SEQ ID NO:94) | |

The present invention provides a method for detecting the presence or absence of CYP2D6 MRNA and control gene MRNA using a detection system such as the ABI PRISM® 7700 Sequence Detection System or the Gene Amp 570 Sequence Detection System. However, the invention further contemplates the use of other detection systems, including, for example, Applied Biosystems GeneAmp® 5700, AMI Prism® 7000 and 7900; Cepheid Smartcycler®; Roche Lightcycler™, BioRad iCycler™, and the Stratagene Mx4000™.

The present invention also contemplates a kit for assessing expression of a CYP2D6 gene in a sample. The kit would be used to quantify CYP2D6 expression to determine interindividual variation of activity of CYP2D6. Further, the kits would be used in selecting or modifying therapy of drugs metabolized by CYP2D6. The kit may comprise, for example: a means for amplifying CYP2D6 mRNA (e.g., primer SEQ ID NO:1 and SEQ ID NO:2) and a labeled compound or agent capable of detecting CYP2D6 MRNA in a sample (e.g., a oligonucleotide probe which binds to nucleic acid encoding CYP2D6, (e.g., SEQ ID NO:3) as well as a means for amplifying a control gene, for example β-GUS (e.g., primer SEQ ID NO:4 and SEQ ID NO:5), and a labeled compound or agent capable of detecting β-GUS mRNA in a sample (e.g., a oligonucleotide probe which binds to nucleic acid encoding β-GUS, (e.g., SEQ ID NO:6). The kit may further comprise, for example: an oligonucleotide, e.g., a detectably labeled oligonucleotide, which hybridizes to a CYP2D6 nucleic acid sequence; or a pair of primers useful for amplifying a CYP2D6 nucleic acid molecule. The kit may also include one of the following: a preservative, a substrate, a control sample or a set of control samples; and instructions.

The calculated ratio of CYP2D6 to β-GUS expression can then be compared to existing data correlating mRNA levels to enzyme activity as a tool for dosage selection of CYP2D6 metabolized drugs for an individual patient. For example, the calculated ratio could be compared to those of normal or control population or population in which given ratio has been correlated to a CYP activity phenotype. This will be particularly useful for pharmaceutical agents or other compounds with a narrow therapeutic index. Rather then have a patient initiate therapy at a fixed recommended dose which will result in lack of efficacy in some patients and risk of toxicity in others, mRNA expression analyses could be performed prior to therapy and based on these results, more or less drug could be administered to improve the rate at which efficacy is achieved while minimizing the risk of toxicity.

### C. Multiplex Test for SNPs of CYP2D6

The present invention also provides a novel method for detecting SNPs of CYP2D6 gene in a biological sample.

We have developed a multiplex test for identifying SNPs of the CYP2D6 gene. As used herein, “multiplex” refers to analysis of two or more targets, including but not limited to SNPs, simultaneously. To perform this test assay, we have developed primers to specific regions of the CYP2D6 gene, probes specific for known SNPs of the CYP2D6 gene; and an identification system for detecting SNPs of the CYP2D6 gene. According to this method, nucleic acid, specifically DNA, is extracted from a biological sample and amplified by known PCR methods. In particular, specific exons of the CYP2D6 gene are amplified using biotinylated primers during the PCR reaction. After PCR amplification, the resulting amplicons are hybridized with labeled probes for SNPs of the CYP2D6 gene and detected using any one of several known identification systems. In one embodiment, we have used fluorescently-dyed microsphere beads (“microspheres” as used herein) that were pre-labeled with oligonucleotide probes specific for SNPs of the CYP2D6 gene. The microspheres have specific ratios of red-emitting and orange-emitting fluorochromes for detection by flow cytometry. After hybridization with the amplicons, the labeled microspheres are then detected by flow cytometry. The results are reported as fluorescent intensities which are converted to ratios of wildtypes to variant for each of the SNPs tested.

An alternative approach to using standard multiplex assay genotyping to screen for mutant alleles is to screen for SNPs within the human CYP2D6 gene using a multiplex assay. The present invention provides a multiplex test to detect of the most common mutant alleles (or SNPs) of CYP2D6 (see Table 3) in a biological sample. This test can be used to screen for the 76 allelic variants that are SNPs. The panel of twelve SNPs used as examples herein were selected on the basis of their overall frequency and their detrimental effects with respect to CYP2D6 enzyme activity. For example, in FIG. 4, we illustrate that the SNP in Exon 6 results in decreased CYP2D6 activity. However, the invention contemplates using the disclosed method to detect the presence of any CYP2D6 SNPs.

We have developed a multiplex assay to detect SNPs in CYP2D6 genes using DNA probes designed for detecting these SNPs under stringent hybridization conditions. Specifically, this method co-amplifies multiple DNA fragments that contain different SNPs of CYP2D6 under optimized conditions followed by detection after hybridization. In a preferred embodiment, the detection system utilizes fluorescently-tagged microspheres. In the preferred embodiment, the presence of a SNPs is detected using flow cytometry, whereby a ratio is generated for each unique SNP.

The criteria used to classify the presence or absence of a specific SNP is preset, i.e., a ratio of at least 3:1 wildtype to SNP is considered to be a homozygous wildtype, while a ratio of 1:1 is a heterozygote and a ratio of 1 at least 3 is a homozygote variant. The classification data for individual SNPs is then entered into an Access database to then predict an individual sample's genotype. For example, a sample classified as heterozygous for SNPs at positions 100, 974, 984, 997, 1846, 2549 and 4180 would be genotyped as CYP2D6*3A/*4B. This subject is therefore a carrier of two variant alleles known to result in a defective CYP2D6 protein with no enzyme activity and would be considered a poor metabolizer. If an individual patient is identified as a poor metabolizer based on the genotype results, this may have several implications. Rather than administration of a CYP2D6 metabolized drug, one option would be to administer a drug metabolized via a different pathway. Alternatively, a lower dose of the drug may be administered. Depending on the agent, this may be 2 fold to 100 fold lower. If a patient is identified by genotyping analysis as an ultrarapid metabolizer, a higher dose of the CYP2D6 metabolized drug would be recommended. If a patient is classified as an extensive or intermediate metabolizer, the current guidelines regarding dosage should be followed and as described above, the mRNA expression analyses could also be performed to better individualize drug therapy.

TABLE 3
CYP2D6

| | Forward primer | Reverse primer | | WT probe | MT probe | Hyb |
|---|---|---|---|---|---|---|
| Exon | -5′biotinylated | -5′biotinylated | SNP | 5′-unilink | 5′-unilink | Temp. |
| 1 | 2D6ex.I Fb | 2D6ex.I Rb | 100 | 5′- | 5′- | 55 |
| | 5′ACTCACAGCAGA | 5′- | | TGCACGCTAC | TGCACGCTACTCA | |
| | GGGCAAAG-3′ | CGAAGCAGTATG | | CCAC | CCAGGCC-3′ | |
| | (SEQ ID NO: 11) | GTGTTCTG-3′ | | CAGGCC-3′ | (SEQ ID NO: 14) | |
| | | (SEQ ID NO: 12) | | (SEQ ID NO: 13) | | |
| 2 | 2D6ex.2Fb | 2D6ex.2Rb | 974 | 5′- | 5′- | 55/56 |
| | 5′- | 5′- | | GCGCGAGGC | GCGCGAGGCG | |
| | GTGATCCTGGCTT | CGGAAATCTGTCT | | GCTGG | ATGGTGACCCA-3′ | |
| | GACAAGAG-3′ | CTGTCC-3′ | | TGACCCA-3′ | (SEQ ID NO: 18) | |
| | (SEQ ID NO: 15) | (SEQ ID NO: 16) | | (SEQ ID NO: 17) | | |
| | | | 1023 | 5′- | 5′- | 55 |
| | | | | GTGCCCATCA | GTGCCCATCATCC | |
| | | | | CCCA | AGATCCTG-3′ | |
| | | | | GATCCTG-3′ | (SEQ ID NO: 20) | |
| | | | | (SEQ ID NO: 19) | | |
| | | | 1039 | 5′- | 5′- | 55/56 |
| | | | | TCCTGGGTT | TCCTGGGTTTTGG | |
| | | | | TCGGG | GCCGCGTT-3′ | |
| | | | | CCGCGTT-3′ | (SEQ ID NO: 22) | |
| | | | | (SEQ ID NO: 21) | | |
| 3,4 | 2D6EX3,4Fb.552 | 2D6EX3,4Rb.552 | 1638 | 5′- | 5′- | |
| | 5′TGGATGGTGGG | 5′TATGCAAATCCT | | GCCCGCGTGG | GCCCGCGTGGCGC | |
| | GCTAATGC-3′ | GCTCTTCCG-3′ | | GGCG | GAGCAGAG-3′ | |
| | (SEQ ID NO: 23) | (SEQ ID NO: 24) | | AGCAGAG-3′ | (SEQ ID NO: 26) | |
| | | | | (SEQ ID NO: 25) | | |
| | | | 1661 | 5′- | 5′- | 55/56 |
| | | | | CGCTTCTCCG | CGCTTCTCCGTCT | |
| | | | | TGTCC | CCACCT-3′ | |
| | | | | ACCT-3′ | (SEQ ID NO: 28) | |
| | | | | (SEQ ID NO: 27) | | |
| | | | 1707 | 5′- | 5′- | 51/52 |
| | | | | GCTGGAGCA | GCTGGAGCAGGG | |
| | | | | GTGGG | GTGACCGA-3′ | |
| | | | | TGACCGA-3′ | (SEQ ID NO: 30) | |
| | | | | (SEQ ID NO: 29) | | |
| | | | 1749 | 5′- | 5′- | 51/52 |
| | | | | GCCTTCGCCA | GCCTTCGCCGACC | |
| | | | | ACCA | ACTCCGGT-3′ | |
| | | | | CTCCGGT-3′ | (SEQ ID NO: 32) | |
| | | | | (SEQ ID NO: 31) | | |
| | | | 1758 | 5′- | 5′- | |
| | | | | CAACCACTCC | CAACCACTCCTGT | |
| | | | | GGTG | GGGTGATG-3′ | |
| | | | | GGTGATG-3′ | (SEQ ID NO: 34) | |
| | | | | (SEQ ID NO: 33) | | |
| | | | 1976 | 5′- | 5′- | 55/56 |
| | | | | GCTCAGGAG | GCTCAGGAGGAAC | |
| | | | | GGACT | TGAAGGAG-3′ | |
| | | | | GAAGGAG-3′ | (SEQ ID NO: 36) | |
| | | | | (SEQ ID NO: 35) | | |
| | 2D6EX3,4Fb.115 | 2D6EX3,4Rb.115 | 1846 | 5′- | 5′- | 55 |
| | 5′- | 5′- | | GCATCTCCCA | GCATCTCCCACCC | |
| | ATGGGCAGAAGG | TTGTCCAAGAGAC | | CCCC | CCAAGACG-3′ | |
| | GCACAAAG-3′ | CGT-TGGG-3′ | | CAGGACG-3′ | (SEQ ID NO: 40) | |
| | (SEQ ID NO: 37) | (SEQ ID NO: 38) | | (SEQ ID NO: 39) | | |
| 5 | 2D6ex.5Fb | 2D6ex.5Rb | 2549 | 5′- | 5′- | 55/56 |
| | 5′- | 5′- | | AACTGAGCAC | AACTGAGCACGGA | |
| | ACTTGTCCAGGT | CTGACCTCCAATT | | AGGA | TGACCTG-3′ | |
| | GAACGCAG-3′ | CTGCACC-3′ | | TGACCTG-3′ | (SEQ ID NO: 44) | |
| | (SEQ ID NO: 41) | (SEQ ID NO: 42) | | (SEQ ID NO: 43) | | |
| 6 | 2D6ex.6Fb | 2D6ex.6Rb | 2850 | 5′- | 5′- | 55/56 |
| | 5′- | 5′- | | TGAGAACCTG | TGAGAACCTGTGC | |
| | TTCTGTCCCGAGT | ACAGGCACCTGCT | | CGCA | ATAGTGGT-3′ | |
| | ATGCTC-3′ | GAGAAAG-3′ | | TAGTGGT-3′ | (SEQ ID NO: 48) | |
| | (SEQ ID NO: 45) | (SEQ ID NO: 46) | | (SEQ ID NO: 47) | | |
| | | | 2938 | 5′- | 5′- | 55/56 |
| | | | | ATCCTACATCC | ATCCTACATCTGG | |
| | | | | GGA | ATGTGCAGC-3′ | |
| | | | | TGTGCAGC-3′ | (SEQ ID NO: 50) | |
| | | | | (SEQ ID NO: 49) | | |
| 7 | 2D6ex.7Fb | 2D6ex.7Rb | 3288 | 5′- | 5′- | 55 |
| | 5′- | 5′- | | CGTCCCCCTG | CGTCCCCCTGAGT | |
| | AGGCAAGAAGGA | TCAGTGTGGTGG | | GGTG | GTGACCCA-3′ | |
| | GTGTCA-3′ | CATTGAG-3′ | | TGACCCA-3′ | (SEQ ID NO: 54) | |
| | (SEQ ID NO: 51) | (SEQ ID NO: 52) | | (SEQ ID NO: 53) | | |
| 8 | 2D6ex.8Fb | 2D6ex.8Rb | 3828 | 5′- | 5′- | 55 |
| | 5- | 5′- | | ACCTGTCATC | ACCTGTCATCAGT | |
| | CAGCATCCTAGA | ACAGGCACCTGCT | | GGTG | GCTGAAGG-3′ | |
| | GTCCGTCC-3′ | GAGAAAG-3′ | | CTGAAGG-3′ | (SEQ ID NO: 58) | |
| | (SEQ ID NO: 55) | (SEQ ID NO: 56) | | (SEQ ID NO: 57) | | |
| | | | 3877 | 5′- | 5′- | 55/56 |
| | | | | CTTCCACCCC | CTTCCACCCCCAA | |
| | | | | GAAC | CACTTCC-3′ | |
| | | | | ACTTCC-3′ | (SEQ ID NO: 60) | |
| | | | | (SEQ ID NO: 59) | | |
| 9 | 2D6ex.9Fb | 2D6ex.9Rb | 4180 | 5′- | 5′- | 45 |
| | 5′- | 5′- | | TGAGCCCATC | TGACCCCATCCCC | |
| | AGTCTTGCAGGG | TCTGCTCAGCCTC | | CCCC | CTATGAG-3′ | |
| | GTATCAC-3′ | AACGTAC-3′ | | CCTATGAG-3′ | (SEQ ID NO: 64) | |
| | (SEQ ID NO: 61) | (SEQ ID NO: 62) | | (SEQ ID NO: 63) | | |
| 5′ | 2D6.PRFb | 2D6.PRRb | −1584 | 5′- | 5- | |
| | 5′- | 5′- | | TtggAAgAACCC | TTggAAgAACgCggT | |
| | TTCAAGACCAGCC | GTGCCACCACGTC | | ggTC | CTCTAC-3′ | |
| | TGGACAAC-3′ | TAGCTTT-3′ | | TCTAC-3′ | (SEQ ID NO: 68) | |
| | (SEQ ID NO: 65) | (SEQ ID NO: 66) | | (SEQ ID NO: 67) | | |

The biological sample to be tested may be collected in any sterile container. Preferably, the sample is whole blood. However, the present method can be applied to any type of biological sample. For example, hepatocytes, leukocytes, and tumor cells are types of sample that may be analyzed for the presence of SNPs using the present invention. The advantages of using whole blood as the sample, instead of other potential samples, are the ease of acquisition, less invasive nature of obtaining the sample, real time data obtained, decreased cost, and fewer processing steps.

Probes specific for CYP2D6 consist of 18-20 mer complementary regions surrounding the SNP. In one embodiment, probes are attached covalently to the surface of fluorescently-dyed microsphere beads. Using this system, oligonucleotides for wild type and mutant sequences are synthesized with a “unilinker” modification at the 5′end (Oligos Etc, Wilsonville, Oreg.). The unilinker is comprised of a one carbon spacer between the oligonucleotide sequence and a reactive amine.

In a preferred embodiment, universally tagged allele specific primers are used instead of individually tagged primers. For example, the Tm Bioscience Tm-100 Universal Sequence Set™ is a commercially available set of 100 unique 24 mer DNA sequences that can easily be adapted for application using the Luminex system. This sequence set facilitates high specificity along with flexibility. This alternative approach requires only a single hybridization optimization step, as opposed to the target specific sequence hybridization optimizations which must be performed for each unique assay. In this embodiment, oligonucleotide probes targeted to specific CYP2D6 SNPs are linked to the universally tagged primers by either ligation or primer extension chemistry and then used in the Luminex multiplexed assay in place of the unilinker oligonucleotide probes.

In a preferred embodiment, genotyping is performed by hybridizing the biotinylated PCR products to microspheres that have been tagged with universal tag primers linked to probes for the CYP2D6 SNPs. Hybridized PCR products are then stained and signals are detected by flow cytometry. Preferably, the flow cytometry is performed by the Luminex 100 instrumentation and software. This same approach can be applied to alternative microsphere systems instrumentation.

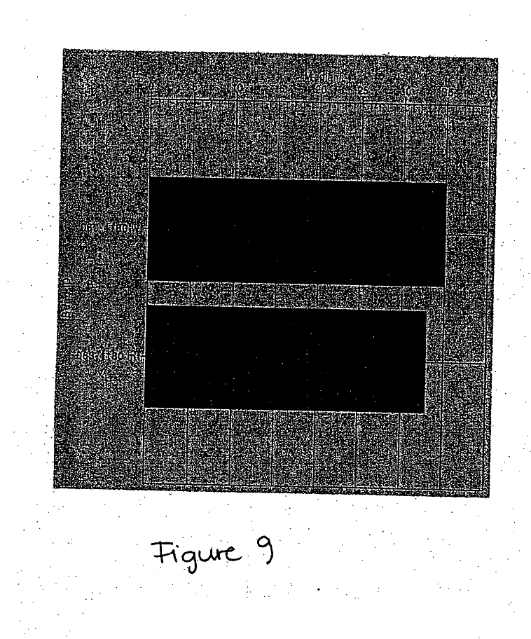

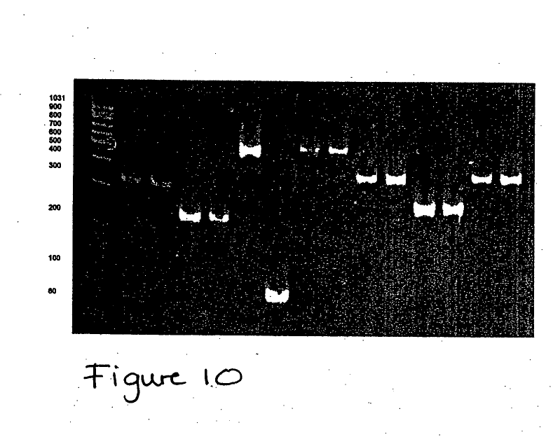

Detection and analysis of the labeled SNPs of CYP2D6 gene is performed using the Luminex multiplex acquisition software. The key data obtained is the relative fluorescence signal obtained for the wildtype and mutant probe for a specific SNP. Data generated for each microsphere is captured in either tabular or graphic form as mean fluorescence intensity units. (see, e.g., FIGS. 8 and 9) The background fluorescence signal from unlabeled microspheres is subtracted. The ratio of mean fluorescence signals for wildtype versus mutant SNP is then calculated. A minimum ratio of 3:1 (or 1:3) is used to define homozygosity for a SNP; otherwise, the sample is classified as a heterozygote. Positive and negative controls are included with each assay. Positive controls are samples for which the genotype has been confirmed by sequencing. Negative controls include PCR negative controls and unlabeled microspheres.

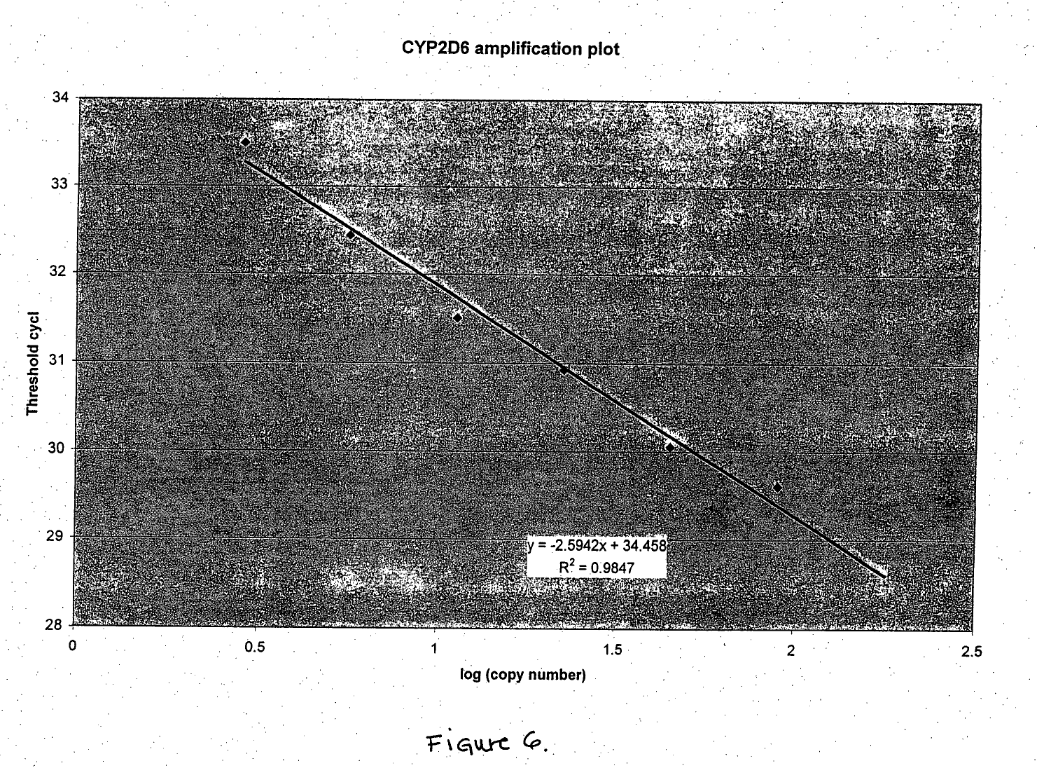

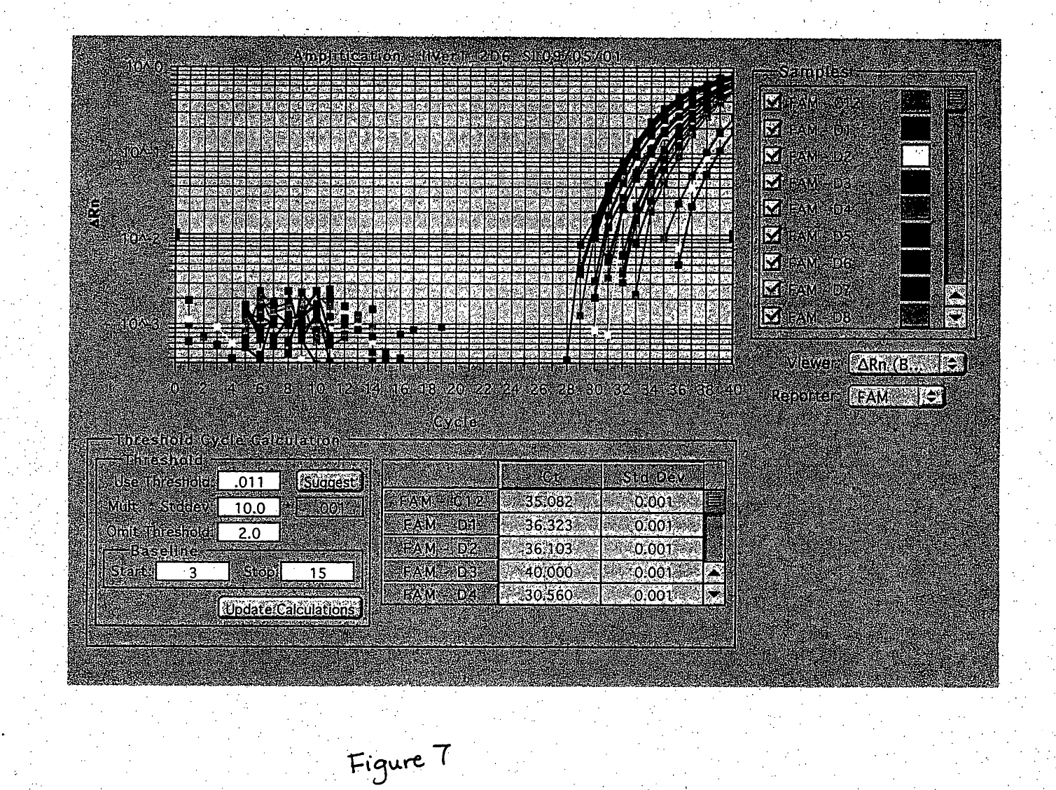

Statistical analysis methods are used to normalize the fluorescence data and provide estimates of variability that correct for potential confounded effects, such as the amount and concentration of PCR product hybridized to the microphere species, probe labeling efficiency, etc. An example of results from a multiplexed reaction screening for 3 distinct CYP2D6 SNPs is illustrated in FIG. 5.

### D. NA TIINA T2 Multiplexed Genotyping Assay

The invention further provides a method for assessing a patient's drug metabolism status as it relates to NAT1 and NAT2 genotypes. In particular, we have developed a qualitative multiplex test to detect SNPs in NAT1 and NAT2 using DNA probes designed for detecting these SNPs under stringent hybridization conditions. (See Table 4). Specifically, this method entails co-amplifying multiple DNA fragments containing different SNPs of NAT1 and NAT2 under optimized conditions, followed by detection using a microchip platform. Table 4 provides a summary of the panel of NAT1/NAT2 SNPs screened, optimal hybridization temperatures for fluorescence, and scanning using the Nanogen workstation, the Nanochip, and the orientation of the PCR product DNA strand screened.

The present invention can be carried out on a nucleic acid sample from any type of biological sample. Preferably, the sample is whole blood. However, the present method can be applied to any type of biological sample, for example, hepatocytes, leukocytes, and tumor cells. However, samples other than whole blood would require more extensive and invasive acquisition procedures and sample processing. Moreover, to increase efficiency of the sample collection and decrease cost, it is preferable to use whole blood.

Following collection of a whole blood sample in a collection tube, preferably a potassium EDTA tube, the whole blood is processed with a lysis reagent. Genomic DNA of the individual sample is isolated by methods known in the art. A number of techniques exist for the purification of DNA from biological samples. Preferably, genomic DNA is isolated from whole blood using the PureGene® DNA isolation kit (Gentra Systems, Minneapolis, Minn.).

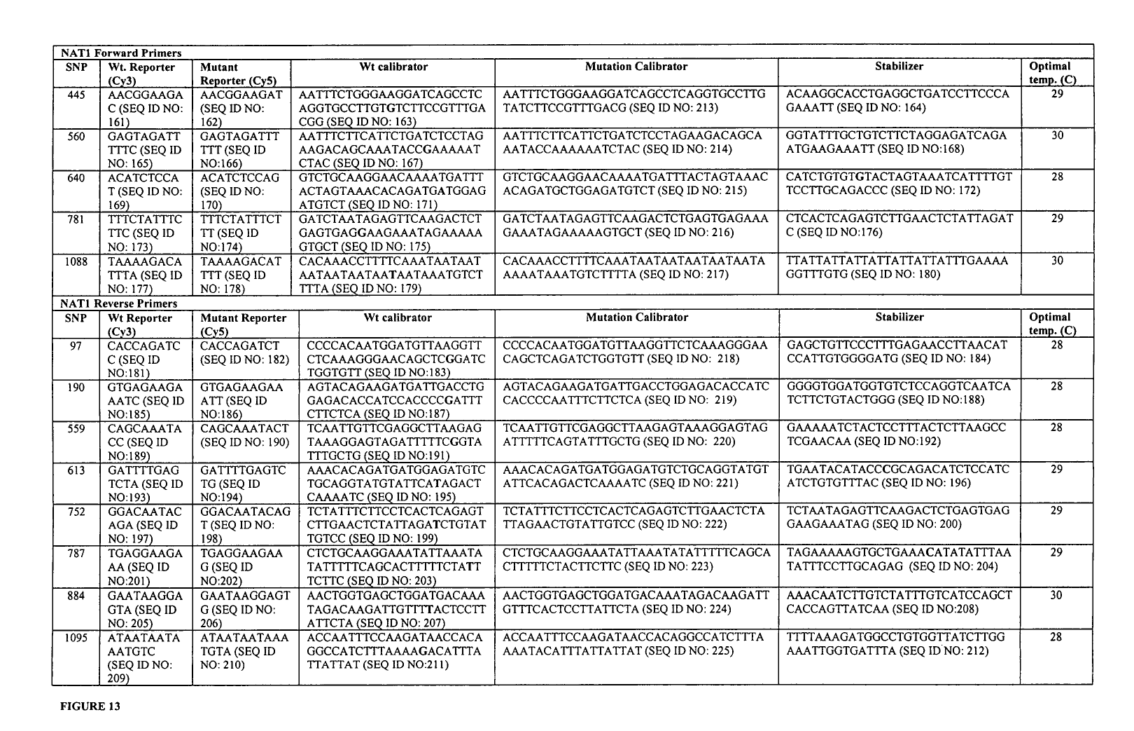

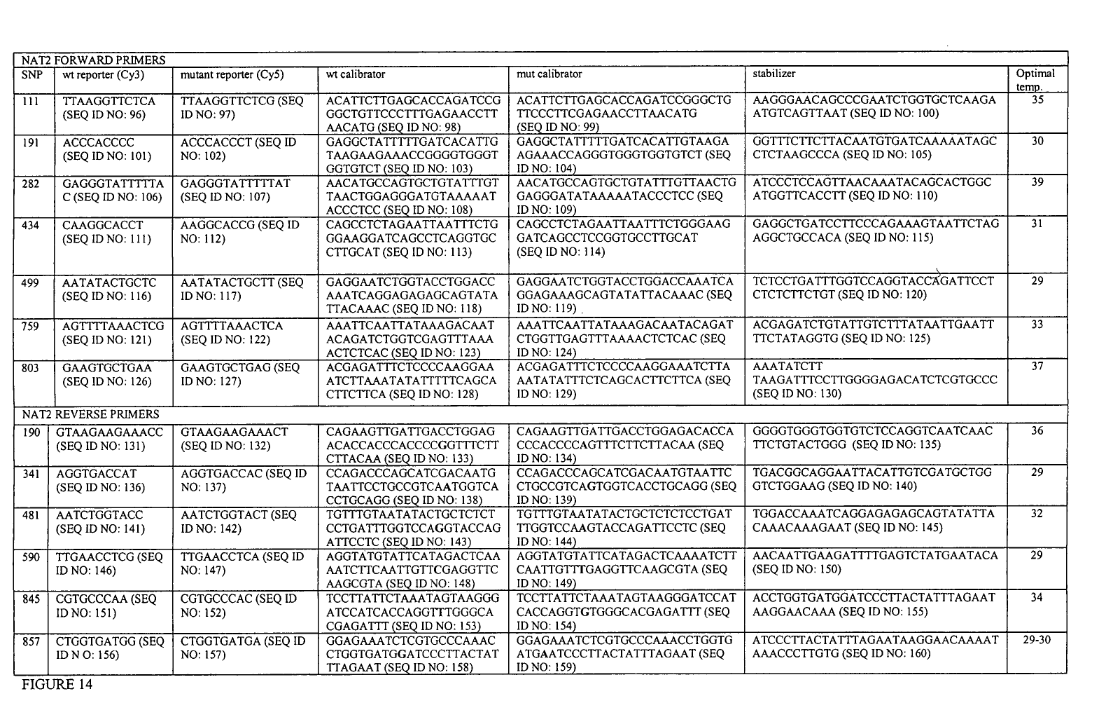

Following the isolation of genomic DNA, the sample is amplified by known protocols. Preferably, the primers used in this assay are tagged with biotin and are designed to amplify the specific exons of NAT1 and NAT2 genes containing the SNPs. In a preferred embodiment, the primers for the amplification of nucleic acid sequence of the NAT 1 and NAT2 genes include, for example, primer pairs SEQ ID NO: 7(5′-ATGGACATTGAAGCATATCT-3′) and SEQ ID NO:8(5′-TGTGGTTATCTTGGAAATTG-3′); or primer pairs SEQ ID NO:9(5′-GGAACAAATTGGACTTG-3′) and SEQ ID NO:10(5′-GCAGAGTGATTCATGCTAGA-3′) for NAT 1 and NAT2, respectively. Additional NATI and NAT2 SNPs primer pair and reporter probe sequences are provided in FIGS. 13 and 14 respectively.

The present invention provides a method of arraying amplified DNA fragments at selected sites on a silicon microchip or microarray. Preferably, the microchip is a Nanochip®, manufactured by Nanogen®. In a preferred embodiment, the Nanogen® electronic addressing technology is utilized to array the DNA fragments at selected sites. For example, the selected sites may be electronically activated with a positive charge, which then serve as locations for hybridization and detection.

Wildtype and SNP DNA reporter probes 5′ end labeled with CY3 and CY5 respectively are then hybridized to the DNA fragment and binding of the fluorescent dye is then scanned utilizing a fluorescence scanner for detection of the report probe. Following the hybridization of a pair of reporter probes and data generation and collection, the probes can be washed and stripped from the bound PCR product and a new pair of reporter probes to a second SNP can be screened. In Table 4, the optional hybridization temperatures for the panel of NAT1 and NAT2 SNPs are listed. The orientation of the biotinylated PCR product strand to be screened is also indicated in Table 4. Similar to the SNP data analysis procedure for the Luminex CYP2D6 assay, the fluorescent signal intensity ratios for individual SNPs are then determined. Here, a ratio of less than 3 will be considered a heterozygote and a ratio equal to or larger than 5 will be considered a homozygote wildtype or variant. A ratio of between 3 and 5 is not classified and the sample should be re-analyzed.

TABLE 4

| | |
|---|---|
| Optimal Hybridization | |
| Temperature (° C.) | Forward/Reverse |

NAT1 SNP

| | | |
|---|---|---|
| 97 | 28 | R |
| 190 | 28 | R |
| 445 | 29 | F |
| 559 | 28 | R |
| 560 | 30 | F |
| 613 | 29 | R |
| 640 | 28 | F |
| 752 | 29 | R |
| 781 | 29 | F |
| 787 | 26 | R |
| 884 | 30 | R |
| 1088 | 30 | F |
| 1095 | 28 | R |

NAT2 SNP

| | | |
|---|---|---|
| 111 | 35 | F |
| 190 | 36 | R |
| 191 | 30 | F |
| 282 | 39 | F |
| 341 | 29 | R |
| 434 | 31 | F |
| 481 | 32 | R |
| 499 | 29 | F |
| 590 | 29 | R |
| 759 | 33 | F |
| 803 | 37 | F |
| 845 | 34 | R |
| 857 | 29 | R |

### EXAMPLES

### EXAMPLE 1

### RNA Isolation from Whole Blood and Small Scale Reverse Transcription

RNA Extracted from Whole Blood by the Following General Procedure:

1. A portion of the whole blood sample, approximately 3 ml, collected in a potassium EDTA tube, was placed in a sterile centrifuge tube.

2. A volume of sterile RBC lysis solution, approximately 9 ml, was added to the whole blood sample and mixed thoroughly and incubated for 5 minutes at room temperature, mixed thoroughly a second time and incubated for an additional 5 minutes at room temperature.

3. The mixture was centrifuged at 15,000×g for one minute and the supernatant was decanted and discarded.

4. The remaining pellet was vortexed vigorously and resuspended in 3 ml of cell lysis solution.

5. A 1 ml aliquot of protein-DNA precipitation solution was added to the pellet and lysate mixture and the tube was inverted 10 times and incubated on ice for 10 minutes.

6. The pellet mixture was centrifuged at 15,000×g for five minutes and the supernatant was collected and placed in a new sterile tube.

7. 3 ml of isopropanol was added to the supernatant and mixed thoroughly before being centrifuged at 15,000×g for five minutes.

8. The supernatant was decanted and the pellet was washed with 3 ml of 70% ethanol and subsequently centrifuged at 15,00×g for 2 minutes.

9. The ethanol was decanted and the pellet was vacuum dried for 5 minutes. The pellet was then resuspended in 25 μl DEPC dH₂O (Sigma).

10. 5 μl of the resulting pellet and dH₂O mixture was further diluted in 995 μl of DEPC dH₂O.

Small scale reverse transcription

1. To a sterile 0.5 ml PCR tube, 0.1 μl of random hexamers (Pharmacia), 0.5 μl RNAsin (Promega), and 10 μg RNA from a patient was added.

2. The total volume was adjusted to be 9.5 μl by adding dH₂O.

3. The mixture was heated at 94° C. for 2 minutes in a PCR thermalcycler.

4. The mixture was chilled on ice for 5 minutes before adding 5 μl RNAsin (Promega), 4 μl 5×MMLV buffer (Gibco, BRL), 2 μl 0.1 M DTT (Gibco, BRL), 2 μl dNTP mix (10 mM mix)(Pharmacia).

5. The mixture was thoroughly mixed and incubated for 15 minutes at 41° C.

6. 2 μl MNLV Superscript II (Gibco BRL) was added and returned to the thermacyler and incubated for the following cycling conditions: 60 minutes at 41° C., followed by 5 minutes at 99° C., and finally 5 minutes at 41° C.

### EXAMPLE 2

### CYP2D6 MRNA Quantitation by Q-PCR

Sample Collection and Processing: RNA was isolated from whole blood as illustrated in Example 1. However, RNA may be isolated by any standard techniques as described in the references cited above.

PCR Ouantitation of mRNA expression: Quantitation of CYP2D6 cDNA and a control gene (e.g., β-GUS) cDNA was carried out using a fluorescence based real-time detection method (ABI PRISM® 7700 or 7900 Sequence Detection System [TaqMan®], Applied Biosystems, Foster City, Calif.) as described by Heid et al., (Genome Res 1996; 6:986-994) and Gibson et al., (Genome Res 1996;6:995-1001), which are hereby incorporated by reference in their entirety. This method uses a dual labeled fluorogenic TaqMan® oligonucleotide probe, (CYP2D (SEQ ID NO:3), Tₘ=70° C.; β-GUS (SEQ ID NO:6)) that anneals specifically within the forward and reverse primers. Laser stimulation within the capped wells containing the reaction mixture causes emission of a 3′quencher dye (TAMRA) until the probe is cleaved by the 5′ to 3′nuclease activity of the DNA polymerase during PCR extension, causing release of a 5′ reporter dye (6FAM). Production of an amplicon thus causes emission of a fluorescent signal that is detected by the TaqMan®'s CCD (charge-coupled device) detection camera, and the amount of signal produced at a threshold cycle within the purely exponential phase of the PCR reaction reflects the starting copy number of the sequence of interest. Comparison of the starting copy number of the sequence of interest with the starting copy number of the control gene provides a relative gene expression level. TaqMan® analyses yield levels that are expressed as ratios between two absolute measurements (gene of interest/ control gene).

The PCR reaction mixture consisted of 0.5 μl of the reverse transcription reaction containing the cDNA, prepared as described, 0.25 μl (or 10 μM) of each oligonucleotide primer CYP2D6.Ftaq (SEQ ID NO:1) and CYP2D6.Rtaq (SEQ ID NO:2), 0.25 μl (10 μM) TaqMan® probe (SEQ ID NO:3), 6.75 μl dH₂O and 12.5 μl 2×TaqMan® Buffer A containing a reference dye, for a final volume of less than or equal to 25 μl (all reagents Applied Biosystems, Foster City, Calif.). Cycling conditions were 50° C. for 2 min, 1 cycle at 95° C. for 12 minutes, 95° C. for 20 seconds followed by 40 cycles at 60° C. for 1 min each. Oligonucleotides used to quantify control gene β-GUS were GUS.Ftaq (SEQ ID NO:4) and GUS.Rtaq (SEQ ID NO:5). The PCR reaction mixture for 13-GUS detection and quantitation consisted 0.5 μl of the reverse transcription reaction containing the cDNA, prepared as described by the method above, 0.25 μl (or 10 μM) of each oligonucleotide primers GUS.Ftaq (SEQ ID NO:4) and GUS.Rtaq (SEQ ID NO:5), 0.25 μl (10 μM) TaqMan® probe (SEQ ID NO:6), 6.75 μl dH₂O and 12.5 μl 2×Taqman® Buffer A containing a reference dye, to a final volume of less than or equal to 25 μl (all reagents Applied Biosystems, Foster City, Calif.). Cycling conditions were 50° C. for 2 min, 1 cycle at 95° C. for 12 minutes, 95° C. for 20 seconds followed by 40 cycles at 60° C. for 1 min each.

Amplification of genomic DNA and pseudogenes, when genomic DNA contaminates RNA samples, may hamper the correct evaluation of RT reaction, yielding amplified products with sizes identical or close to the target specific amplicons. To avoid this problem, in our experiments, we have treated the isolated total RNA samples with RNase free DNase to digest potentially contaminating genomic DNA, and have designed primers to span an intron/exon junction in order to PCR amplify cDNA product only. Further, we have selected a primer pair that does not amplify the CYP2D7 and CYP2D8 pseudogenes, verified by running a nucleotide BLAST search on the internet at http://www.ncbi.nlm.nih.gov/BLAST/.

The primers used to amplify the CYP2D6 gene from human are shown in Table 1. Primers were designed based upon publically available cDNA and intron/exon boundary sequence.

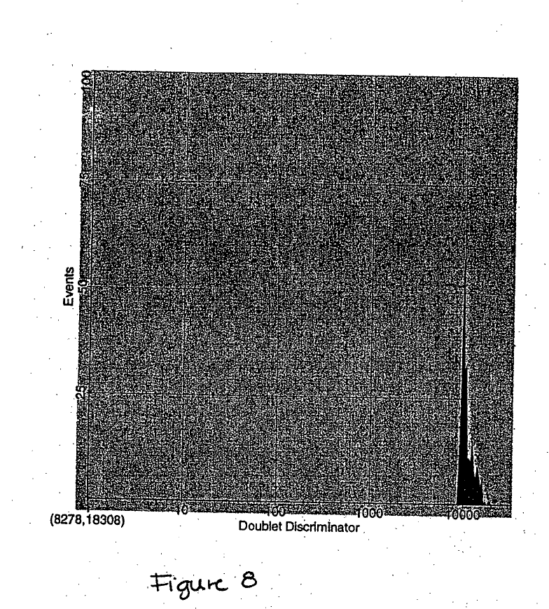

Results and Analysis: FIG. 7 shows the quantitative results for a series of diluted cDNA samples. The PCR efficiencies are calculated from the slope of a standard curve of a serial dilution of cDNA template, for which CT is plotted versus the log cDNA concentration from the data collected as shown in FIG. 7. Most gene expression analysis studies using TaqMan® utilize a relative expression calculation similar to standard assays such as a Northern blot. Expression of the gene of interest is reported relative to expression of an endogenous control gene, which is assumed to have equal expression in all tissues in the study. In this way, expression levels can be compared from sample to sample. In TaqMan® assays, relative expression of MRNA species is calculated using a comparative C_{T} method as described previously (See Collins, et al., Proc. Natl Acad. Sci. U.S.A., 95:8703-8709, 1998; and PE Applied Biosystems user bulletin #2., 1997) and briefly below.

Relative Expression Calculations with TagMan®: In the following description, the term “reference” is used to indicate a reference RNA such as β-GUS and the term “calibrator” indicates a sample RNA used as a common denominator for comparative results. For each RNA sample (including the calibrator sample), a difference in C_{T} values (ACT) is calculated for each mRNA by taking the mean C_{T} of duplicate PCR reactions and subtracting the mean C_{T} of the duplicate reactions for a reference RNA measure in an aliquot from the same RT reaction.

ΔC_{T}=C_{T}(test gene)−C_{T}(reference gene). {Eq. 1}

The ΔC_{T} for the calibrator sample is then subtracted from the ΔC_{T} for the test sample to generate a ΔΔC_{T}.

ΔΔC_{T}=ΔC_{T}(test RNA)−ΔC_{T}(calibrator RNA). {Eq. 2}

Preferably, three reverse transcription reactions are carried out for each analysis using different amounts of RNA in each reaction (typically 400, 200 and 100 ng total RNA). This results in a more reliable estimate of gene expression than doing a single RNA input several times. (See Collins and PE Bulletin supra). Thus, 3 such ΔΔC_{T} measurements are obtained for each RNA sample (ΔΔC_{T}(400 ng), ΔΔC_{T}(200 ng) and ΔΔC_{T}(100 ng)). The mean of these ΔΔC_{T} measurements is then used to calculate expression of the test gene, relative to the reference gene and normalized to the calibrator sample as follows:

Relative Expression=(1+E)^{-meanΔΔCT} {Eq. 3}

Where E is the PCR efficiency calculated according to the following formula:

E=10^{(l/S)}−1 {Eq. 4}

Where S equals the slope of a standard curve of a serial dilution of cDNA template, for which C_{T} is plotted versus the log cDNA concentration. PCR efficiency is measured as part of the validation of all new TaqMan® assays and is usually (and is required to be)>95% when the amplicon is designed with strict parameter limitations. Thus, for simplicity, efficiency is assumed to be 100% and therefore:

Relative Expression=2^{-mean ΔΔCT}

The calculated ratio of CYP2D6 to β-GUS expression can then be compared to existing data obtained in a healthy or control population correlating MRNA levels to enzyme activity as a tool for dosage selection of CYP2D6 metabolized drugs for an individual patient. Based on the value of this comparative ratio in the healthy population distribution, we have predicted dose modifications. (See Carcillo J A, et al. Coordinated intrahepatic and extrahepatic regulation of cytochrome P4502D6 in healthy subjects and in patients after liver transplantation Clin Pharm and Ther 2003; 73 (5):456-467).

### EXAMPLE 3

The application of MRNA quantitation of CYP2D6 to guide individualized drug therapy can be exemplified by a patient who presents with an acute psychosis in whom the atypical antipsychotic drug risperidone is indicated for therapeutic management. The therapeutic conundrum in conventional approaches to therapy is balancing the clinical need to achieve rapid control of symptoms with the wide range in dosage requirements between patients together and the risk of tardive dyskinesia and other CNS side effects if the dosage is inappropriately high. Until the advent of the present invention, the routine in clinical practice has been conservative and therapeutic dose range slowly in order to minimize adverse drug events affecting the large proportion of patients who require higher doses, i.e., those who remain hospitalized and symptomatic for longer periods.

Risperidone is exclusively metabolized by CYP2D6. Variations in the rate of metabolism are responsible for differences in dose requirement. With the advent of this new invention, a blood sample drawn at the time of diagnosis can be used to measure the mRNA concentration and predict in vivo CYP2D6 activity, and from this determine whether a high, medium or low dose should be used at the outset of therapy. In this instance, response is predominantly related to parent drug; thus, slow metabolizers require a low dose, normal metabolizers a normal dose and fast metabolizers a high dose to maintain similar levels of parent drug. This ability to individualize therapy at the outset provides an optimal potential to rapidly and effectively control psychosis while minimizing drug induced adversity.

### EXAMPLE 4

A further illustrative example in the application of mRNA quantitation of CYP2D6 to guide individualized drug therapy is a patient with primary hepatocellular cancer who requires therapy infused directly into the tumor via the hepatic artery. In such cases, patients require pain therapy for two separate indications which are characterized by different characteristics and time courses. In both instances oxycodone is the therapy of choice, and in both instances individual dose requirements vary considerably.

The pain associated with intraheptic artery administration of therapy induces an acute peritoneal inflammation that causes severe to disabling pain lasting for approximately 6-12 hours. On the other hand, the pain associated with tumor growth is a deep-seated persistent ache that persists and substantially detracts from the quality of life.

The major problem in administering oxycodone or long-acting OxyContin® for pain management is that with the interindividual variation in dose requirement, a standard dose results in a subtherapeutic effect in a proportion of patients, an optimal response in a proportion of patients, but excessive sedation, constipation and even respiratory depression in others. Oxycodone is metabolized by CYP2D6 to a metabolite that has ten times the activity of the parent compound. Thus, CYP2D6 activity is a major determinant of circulating analgesic. In this instance, measurement of mRNA for CYP2D6 prior to the administration of drug will permit identification of slower metabolizers who require a high dose, normal metabolizers who require a normal dose and fast metabolizers who require a low dose. Thus, the dose modification paradigm is dependent on whether parent drug or metabolite is the active moiety.

The application of the proposed invention permits optimization of pain management for both acute and chronic pain syndromes not only for patients with hepatocellular cancer, but other cancer related pain syndromes.

### EXAMPLE 5

Qualitative Testing for SNPs of CYP2D6-Genotyping Using the Microsphere and Flow Cytometry Technology

The following summarizes the procedures used in the application of microsphere and flow cytometry technology for detection of SNPs of CYP2D6 gene.

Sample Collection and Processing: Whole blood was collected in a collection tube. Genomic DNA was isolated from whole blood by using the PureGene® DNA isolation kit. However, any of the standard methods known in the art for DNA isolation and as described in the cited references may be used for DNA isolation.

DNA isolation: Genomic DNA was isolated from whole blood using the PureGene® DNA isolation kit (Gentra Systems, Minneapolis, Minn.). DNA concentration was measured by determining absorbance at 260 nm on a Shimadzu UV-2101 PC spectrophotometer. Ratios at 260/280 nrn were used to estimate DNA purity, i.e., contamination with protein and samples were used at 260/280 ratios of at least 1.75.

PCR Reaction: Specific exons 3-4 splice mutation (*4B) of CYP2D6 were amplified using biotinylated primers in the PCR reaction. The primer sequences chosen were based on the published sequence of the human CYP2D6 gene. All primers were designed using MacVector™ 4.1.4 to have annealing temperatures of 55/56° C. For large scale yields of PCR product for assay development, PCR was performed using 1 μl template DNA, 1.0 μl each of forward and reverse biotinylated primers (2D6EX3,4Fb.552 and 2D6EX3.4Rb.552), and 0.5 μl Rtaq Gold® DNA polymerase (Applied Biosystems) in a 100 μl reaction. Specifically, the PCR reaction mixture for 3-4 splice mutation (*4B) CYP2D detection consisted of 1.0 μl of the template DNA, 10 μl DMSO, 8.0 μl 25 mM MgCl₂, 2.0 μl (10 mM) dNTP mix, 1.0 μl each of forward and reverse biotinylated primers (ZH1 and ZH2), 0.5 μl AmpliTaq Gold Polymerase, and 66 μl dH₂O. Cycling conditions were 94° C. for 30 seconds, 57° C. for 30 seconds, and 40 cycles at 72° C. for 30 seconds each cycle, followed by a 7 minute extension at 72° C. followed by a 4° C. soak.

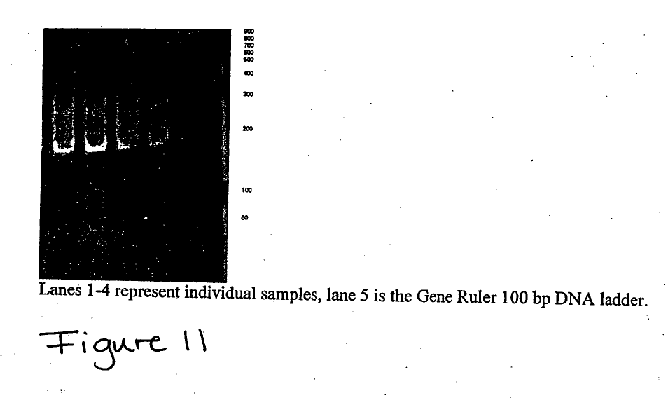

The sample is run on an 8% acrylamide gel at 250 V for approximately 2 hours and stained in ethidium bromide (10 μl/100 ml dH₂O) for 30 minutes. Finally, the gel is destained in clean dH₂O for approximately 30 minutes before photographing the gel. See FIG. 10. According to FIG. 10, the polyacrylamide gel electrophoresis illustrates single exon PCR products for the Luminex based CYP2D6 analyses. Lane 1 is the GeneRuler 100 bp DNA ladder, lanes 2 and 3 exon 1 (329 bp), lanes 4 and 5 exon 2 (247 bp), lane 6 exons 3, 4 (873 bp), lane 7 exons 3, 4 (115 bp), lanes 8 and 9 exon 3, 4 (873 bp), lanes 10 and 11 exon 5 (377 bp), lanes 12 and 13 exon 3 (284 bp), and lanes 14 and 15 exon 7 (382 bp). Data not shown is the exon 9 (212 bp) product.

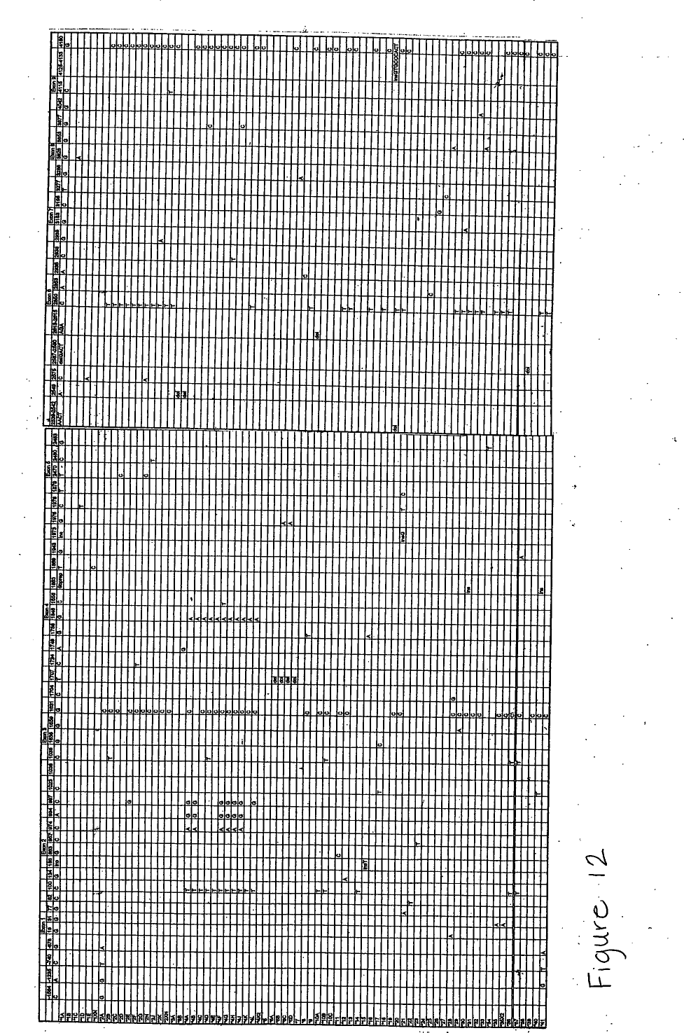

The results for multiplexed PCR for exons 2, 6 and 8 are illustrated in FIG. 11, where lanes 1-4 represent individual samples and lane 5 is the Gene Ruler 100 bp DNA ladder.

Second PCR Reaction: Specific exons 1, 2, 3/4, 5, 6, 8 and 9 were amplified using biotinylated primers in the PCR reaction. Forward and reverse primers were synthesized by Oligos Etc. (Wilsonville, Oreg.) based on the published sequence of the human CYP2D6 gene. All primers were designed using MacVector™ 4.1.4 to have annealing temperatures of 57° C. For large scale yields of PCR product for assay development, PCR was performed using 1 μl template DNA, 1.0 μl each of forward and reverse biotinylated primers (see Table 3 for primer sequence list) and 0.5 μl Rtaq Gold® DNA polymerase (Applied Biosystems) in a 100 μl reaction. Specifically, the PCR reaction mixture for the CYP2D6 exons consisted of 1.0 μl of the template DNA, 10 μl DMSO, 4.0 μl 25 mM MgCl₂, 2.0 μl (10 mM) dNTP mix, 1.0 μl each of forward and reverse biotinylated primers (as provided in Table 3), 0.5 μl gelatin, 0.5 μl AmpliTaq Gold Polymerase, and 70 μl dH₂O. Cycling conditions were 94° C. for 30 seconds, 57° C. for 30 seconds, and 72° C. for 30 seconds for 40 cycles followed by a 7 minute extension at 72° C. followed by a 40 C soak. All exons were amplified under the same conditions to incorporate multiplex PCR reaction.

Each exon's PCR product was sequenced to verify specificity for CYP2D6. The samples were run on an 8% acrylamide gel at 250 V for approximately 2 hours and stained in ethidium bromide (10 μl/100 ml dH₂O) for 30 minutes. Finally, the gel was destained in clean dH₂O for approximately 30 minutes before photographing the gel. (See FIG. 10). The sizes of the amplified fragments were: exon 1 (329 bp), exon 2 (247 bp), exons 3, 4 (873 bp), exon 5 (377 bp), exon 6 (284 bp), and exon 7 (382 bp).

Alternatively, individual exon PCR products were generated which incorporate the universal tag sequences.

Microsphere Labeling: Luminex Corporation (Austin, Texas) manufactured 5.5 μm diameter polystyrene microspheres that have specific ratios of red-emitting (>650 nm) and orange-emitting (585 nm) fluorochromes. Currently, 100 distinct microsphere species with specific ratios of red to orange fluorescence permit their discrimination by flow cytometry and are available. The microspheres are coated with carboxyl groups to permit the covalent coupling of any probe that has been modified with a reactive amine. There are 1-2×10⁶ binding sites per microsphere. Probes specific for CYP2D6 SNPs were designed to be 18-20 bases in length with the SNP located in the center. Oligonucleotides for wild type and mutant sequences were synthesized with a “unilinker” modification at the 5′ end (Oligos Etc, Wilsonville, Oreg.). The unilinker is comprised of a one carbon spacer between the oligonucleotide sequence and a reactive amine. Alternatively, the probes specific for CYP2D6 SNPs may be linked to TM Bioscience Universally-tagged Primers.

The oligonucleotide probe was diluted in sterile distilled water to a final concentration of lnmole/μl.

A 50 μl aliquot of microspheres was brought to room temperature and dispersed by sonication. One nmole probe was added per 5 million micropsheres. 1-Ethyl-3-(3-deimethylaminopropyl) carbodiimide-HCL (EDC, Pierce, Rockville Ill.) was prepared at 10 mg/ml and a 2.5 μl aliquot was added to the microspheres. The microsphere mixture was then gently vortexed. After the mixture was incubated for 30 minutes at room temperature in the dark, the process was repeated with another 2.5 μl aliquot of EDC. Finally, the microspheres were washed twice, first in a 0.02% Tween 20 solution and then in a 0.01% SDS. The beads were centrifuged for one minute at 8,000 ×g before resuspending in 50 μl 0.1 M MES. The beads were enumerated using a hemacytometer and stored in the dark at 4° C.

Hybridization: Hybridization reactions linking the labeled microspheres to PCR product were performed according to manufacturer recommendations in tetramethylammonium chloride (TMAC) buffer, which minimizes the effects of base composition on hybridization rates. For example, a reaction consisted of PCR product up to 17 μl volume which was denatured at 100° C. for 10 minutes on a heat block. Microspheres, pre-labeled with oligonucleotide probe (2500 per SNP per assay) were mixed in 33 μl of 1.5 ×tetramethlyammonium chloride buffer and placed at 58° C. until use. While PCR products were on the heat block, the cap was opened and 33 μl of the microsphere mixture was added. The tube was immediately removed and placed in a heat block set at the appropriate hybridization temperature. After hybridization, microspheres in the samples were collected by centrifugation and incubated with streptavidin-phycoerythrin for at least 5 minutes before fluorescence was measured using the Luminex 100. Beads (coupled with unique probes) were classified according to unique ratios of two classification dyes and data was collected for only those signals appearing in the appropriate windows. Fluorescence associated with each microsphere species was reported separately.

Results and Analysis: As disclosed in FIGS. 8 and 9, the criteria used to classify the presence or absence of a specific SNP is preset. Preferential binding (greater than 3:1 ratio) to either wildtype or variant probe distinguishes a homozygous genotype while equal binding to both wildtype and allelic variant probes identifies heterozygous samples. The information collected for a panel of SNPs is summarized for a given sample and used to generate the genotype classification. The classification data for individual SNPs is then entered into an Access database to then predict an individual sample's genotype. For example, a sample classified as heterozygous for SNPs at positions 100, 974, 984, 997, 1846, 2549 and 4180 would be genotyped as CYP2D6*3A/*4B. This subject is therefore a carrier of two variant alleles known to result in a defective CYP2D6 protein with no enzyme activity and would be considered a poor metabolizer. If an individual patient is identified as a poor metabolizer based on the genotype results, this may have several implications. Rather than administration of a CYP2D6 metabolized drug, one option would be to administer a drug metabolized via a different pathway. Alternatively, a lower dose of the drug may be administered. Depending on the agent, this may be 2 fold to 100 fold lower. If a patient is identified by genotyping analysis as an ultrarapid metabolizer, a higher dose of the CYP2D6 metabolized drug would be recommended. If a patient is classified as an extensive or intermediate metabolizer, the current guidelines regarding dosage should be followed and as described above, the MRNA expression analyses could also be performed to better individualize drug therapy.

### EXAMPLE 6

### The following summarizes the procedures used in the test for NAT1/NAT2 SNPs Using a Microchip Platform.

DNA isolation: Genomic DNA was isolated from whole blood using the PureGene® DNA isolation kit (Gentra Systems, Minneapolis, Minn.). DNA concentration was measured by determining absorbency at 260 mn on a Shimadzu UV-2101 PC spectrophotometer. Ratios at 260/280 nrn were used to estimate DNA purity, i.e., contamination with protein and samples were used at 260/280 ratios of at least 1.75.

PCR Reaction: Specific regions of the NAT1/NAT2 genes were amplified using specific primers for NAT 1 or NAT2 in the PCR reaction by known methods. Forward (SEQ ID NOs: 7 and 9) and reverse primers (SEQ ID NOs:8 and 10) were used.

Sample Preparation: Amplified DNA sample products were desalted in a millipore 96-well filtration unit by adding 50 μl of sample to a well in the filtration plate, followed by adding 50 μl of dH₂O. After allowing the product to filter for 5 minutes, 100 μl of dH₂O is added to each well and allowed to filter an additional 5 minutes. A final aliquot of 60 μl of dH₂O is added to each well and the filtration unit is shaken on a orbital shaker for 5 minutes. The desalted sample products are then carefully transferred to a 96-well plate for loading on a Nanochip.

NanoChip Loading Operation: The amplified and desalted sample products were prepared for electronic addressing by mixing 30 μl of sample product with 30 μl of histidine (100 mM). If a heterozygous sample is not used, a heterozygous calibrator is prepared by mixing 0.5 nM wildtype (wt) and 0.5 mM mutation (mut) calibrator in 50 mM histidine for a final concentration of 0.5 mM. A 60 μl aliquot of the histidine sample reaction solution is added to each well of a 96-well plate. The histidine (as a background control) is addressed using a capture submap format (2.0 V for 60 seconds) and the heterozygous calibrator is addressed using a target submap format (2.0 V for 120 seconds). Finally, the samples are addressed using a target submap format (2.0 V for 180 seconds). The pre-treated Nanogen cartridge (as per manufacturer's suggestion) is washed 3 times with 150 μl of dH₂O followed by five washes with 150 μl of high salt buffer.

NanoChip Reader Operation: The reporters and stabilizer are diluted in high salt buffer according to optimized conditions. After incubating the cartridge for 3 minutes at room temperature, the reporter mixture is removed from the cartridge by washing twice with 150 μl of high salt buffer. A final 150 μl of high salt buffer is added to the cartridge before reading the cartridge in the NanoChip reader. At temperature 24° C., the cartridge is scanned at medium gain. Finally, the NAF file is set according to optimization conditions (see Table 4) and the cartridge is scanned at medium gain. If the values are saturated, lower the pmt accumulation. The cartridge is denatured by NaOH to report the cartridge again. Finally, data analysis is performed using the heterozygous calibrator for normalization of data.

Results and Analysis: The first step in data analysis requires the user to select a capture label (histidine) for background subtraction. The second step comprises of assigning wildtype and mutant status to the respective reporter oligonucleotides. Every cartridge requires a known heterozygote in order to normalize red and green lasers and the difference in hybridization efficiency between green (wildtype) and red(variant) dye labeled reporter oligonucleotides. After the heterozygote control is assigned to a particular sample or to a synthesized heterozygote calibrator, data analysis can be carried out. The criteria to designate genotypes are predefined as described previously. A ratio of less than 3 will be considered a heterozygote, between 3 and 5 is no designation and equal to or larger than 5 will be considered homozygote wildtype or variant. The classification data for an individual SNP is then entered into an Access database to then predict an individual sample's NAT1/NAT2 genotype. For example, a sample classified as a homozygous variant for individual SNPs at positions NAT2 341, 481 and 803 would be genotyped as NAT2*5B/*5B. This subject is therefore a carrier of two variant alleles known to be associated with the slow acetylator phenotype. Individuals genotyped as slow acetylators should either not receive a NAT2 or NAT1 metabolized agent, or be given a reduced dose. For certain drugs, the fast acetylators may also be at increased risk of adverse drug reactions, so again knowledge of the genotype can predict who should be more closely monitored and whether or not the dose should be adjusted.

### EXAMPLE 7

An illustrative example for the application of genotyping of N-acetyltransferase 1 (NAT 1) an N-acetyltransferase 2 (NAT2) is illustrated by its ability to be used in cancer prevention related to long-term employment in industries that expose their workers to procarcinogens. We and other researchers have shown that there is a synergistic risk between certain industrial occupations and the presence of a slow acetylation phenotype for the development of bladder cancer later in life. This information has been obtained by assessing the metabolism of a probe drug such as dapsone, which is a substrate for NAT1 and NAT2. The increase in risk is substantial in the order of 30 fold, but the previously used end-point measure is somewhat cumbersome and requires an invasive method to test. The availability of the NAT1/NAT2 genotyping permits identification of all the known variant alleles for these two genes and prediction of the NAT phenotype from a single blood sample.

The availability of this invention permits an individualized safety assessment for a worker applying to position in a dye making industry with a view to prevention of bladder cancer in the future. If the worker is identified as a predicted slow acetylator, both the worker and the company will be able to appraise long-term risk and take steps to minimize the development of bladder cancer in that individual.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

What is claimed is:

1. A method for measuring the expression of a CYP enzyme in a subject, comprising measuring the expression of the CYP enzyme gene in whole blood.

2. The method of claim 1, wherein the CYP enzyme is selected from the group consisting of CYP1A1, CYP1A2, CYP1B1, CYP2C8, CYP2C9, CYP2C18, CYP2C19, CYP2D6, CYP2E1, CYP3A4 and CYP3A5.

3. The method of claim 1, wherein the CYP enzyme gene expression is measured by measuring the expression of MRNA for the CYP enzyme.

4. The method of claim 3, wherein the method further comprises normalizing the mRNA measurement for the CYP enzyme.

5. The method of claim 4, wherein the normalization comprises comparing the measured expression of the CYP enzyme gene to the expression of a control gene.

6. The method of claim 5, wherein the control gene is β-GUS.

7. The method of claim 1, wherein the CYP enzyme is CYP2D6.

8. A method for measuring the expression of a CYP enzyme in a subject, comprising measuring the expression of the CYP enzyme gene in whole blood and normalizing the measured CYP enzyme gene expression.

9. A method for measuring the activity of a CYP enzyme in a subject, comprising measuring mRNA expression for the CYP enzyme in whole blood and normalizing the measured CYP enzyme mRNA expression.

10. The method of claim 9, wherein the CYP enzyme is selected from the group consisting of CYP1A1, CYP1A2, CYP1B1, CYP2C8, CYP2C9, CYP2C18, CYP2C19, CYP2D6, CYP2E1, CYP3A4, and CYP3A5.

11. A method for measuring CYP enzyme expression in a sample comprising:
(a) isolating and reverse transcribing RNA from the sample to obtain a transcribed product;
(b) subjecting the transcribed product to amplification to obtain an amplified product;
(c) determining the amount of CYP transcribed product in said amplified product; and
(d) comparing the determined amount of CYP transcribed product to a determined amount of transcribed product for a control gene.

12. The method of claim 11, wherein the control gene is selected from the group consisting of βactin, βglucuronidase (β-GUS), glyceraldehyde-3-phosphate dehydrogenase (GAPDH), 18S ribosomal RNA (rRNA), P2-microglobulin, acidic ribosomal protein, cyclophilin, phosphoglycerokinase, hypoxanthine ribosyl transferase, and transcription factor IID (TATA binding protein).

13. The method of claim 10, wherein the sample is a whole blood sample.

14. The method of claim 13, wherein the amplification is by PCR.

15. The method of claim 14, wherein the CYP enzyme is CYP1A1.

16. The method of claim 14, wherein the CYP enzyme is CYP1A2.

17. The method of claim 14, wherein the CYP enzyme is CYP1B1.

18. The method of claim 14, wherein the CYP enzyme is CYP2C8.

19. The method of claim 14, wherein the CYP enzyme is CYP2C9.

20. The method of claim 14, wherein the CYP enzyme is CYP2C18.

21. The method of claim 14, wherein the CYP enzyme is CYP2C19.

22. The method of claim 14, wherein the CYP enzyme is CYP2D6.

23. The method of claim 14, wherein the CYP enzyme is CYP2E1.

24. The method of claim 14, wherein the CYP enzyme is CYP3A4.

25. The method of claim 14, wherein the CYP enzyme is CYP3A5.

26. The method of claim 15, wherein the PCR amplification utilizes primers selected from the group consisting of SEQ ID NO:69 and SEQ ID NO:70, or oligonucleotides substantially identical thereto.

27. The method of claim 16, wherein the PCR amplification utilizes primers selected from the group consisting of SEQ ID NO:72 and SEQ ID NO:73, or oligonucleotides substantially identical thereto.

28. The method of claim 17, wherein the PCR amplification utilizes primers selected from the group consisting of SEQ ID NO:75 and SEQ ID NO:76, or oligonucleotides substantially identical thereto.

29. The method of claim 18, wherein the PCR amplification utilizes primers selected from the group consisting of SEQ ID NO:78 and SEQ ID NO:79, or oligonucleotides substantially identical thereto.

30. The method of claim 19, wherein the PCR amplification utilizes primers selected from the group consisting of SEQ ID NO:81 and SEQ ID NO:82, or oligonucleotides substantially identical thereto.

31. The method of claim 20, wherein the PCR amplification utilizes primers selected from the group consisting of SEQ ID NO:84 and SEQ ID NO:85, or oligonucleotides substantially identical thereto.

32. The method of claim 21, wherein the PCR amplification utilizes primers selected from the group consisting of SEQ ID NO:87 and SEQ ID NO:88, or oligonucleotides substantially identical thereto.

33. The method of claim 22, wherein the PCR amplification utilizes primers selected from the group consisting of SEQ ID NO:1 and SEQ ID NO:2, or oligonucleotides substantially identical thereto.

34. The method of claim 23, wherein the PCR amplification utilizes primers selected from the group consisting of SEQ ID NO:90 and SEQ ID NO:91, or oligonucleotides substantially identical thereto.

35. The method of claim 24, wherein the PCR amplification utilizes primers selected from the group consisting of SEQ ID NO:93 and SEQ ID NO:94, or oligonucleotides substantially identical thereto.

36. A method for measuring CYP2D6 expression in a sample, comprising:
(a) isolating and reverse transcribing RNA from the sample to obtain a transcribed product;
(b) subjecting the transcribed product to amplification; and
(c) determining the amount of CYP2D6 amplified product.

37. The method of claim 35, wherein the sample is whole blood.

38. The method of claim 35, wherein the amplification is by PCR.

39. The method of claim 35, wherein the amount of CYP2D6 amplified product is determined using TAQMAN® analysis.

40. The method of claim 37, wherein the PCR amplification utilizes primers selected from the group consisting of SEQ ID NO: 1 and SEQ ID NO: 2, or oligonucleotides substantially identical thereto.

41. The method of claim 35, further comprising the step of comparing the determined amount of CYP2D6 transcribed product to a determined amount of transcribed product for a control gene.

42. The method of claim 40, wherein the control gene is β-GUS.

43. An oligonucleotide primer having the sequence of SEQ ID NO: 1, or a sequence substantially identical thereto.

44. An oligonucleotide primer having the sequence of SEQ ID NO: 2, or a sequence substantially identical thereto.

45. A kit for detecting expression of CYP2D6 comprising the oligonucleotide of SEQ ID NO: 1 and the oligonucleotide of SEQ ID NO: 2, or oligonucleotides substantially identical thereto.

46. A kit for detecting expression of CYP2D6 comprising an oligonucleotide primer pair for CYP2D6 consisting of SEQ ID NO: 1 and SEQ ID NO: 2 or an oligonucleotide primer pair substantially identical thereto and an oligionucleotide primer pair for the β-GUS gene.

47. A method for detecting SNPs of the CYP2D6 gene in a sample comprising:
(a) isolating DNA from the sample;
(b) subjecting the DNA to amplification; and
(c) subjecting the amplified sample to microspheres labeled with oligonucleotide probes for CYP2D6 SNPs.

48. The method of claim 47, wherein the microspheres are labeled with the probes using universally tagged primers.

49. The method of claim 47, wherein the microspheres are labeled with the probes using a unilinker.

50. The method of claim 47, wherein the method further comprises the step of detecting the presence of labeled CYP2D6 SNPs.

51. The method of claim 47, wherein the sample is whole blood.

52. The method of claim 50, wherein the detection is by flow cytometry.

53. The method of claim 47, wherein the SNPs comprise any one or more of the SNPs listed in FIG. 12.

54. The method of claim 47, wherein the SNPs comprise any one or more of the SNPs listed in Table 3.

55. A method for determining a patient's therapeutic regimen for a drug metabolized by a CYP enzyme comprising:
(a) obtaining a biological sample from the patient;
(b) isolating and reverse transcribing RNA from said sample to obtain transcribed product;
(c) subjecting the transcribed product to amplification using a pair of oligonucleotide primers capable of amplifying a region of the gene for the CYP enzyme to obtain an amplified product;
(d) determining the amount of amplified product;
(e) normalizing the amount of CYP gene amplified product; and
(f) selecting a therapeutic regimen based on the normalized amount of CYP gene amplified product.

56. The method of claim 55, wherein selecting a therapeutic regimen comprises comparing the normalized amount of CYP gene amplified products to the measured CYP gene amplified products for control subjects or a control population.

57. The method of claim 56, wherein the measured amount of CYP gene amplified product for the control subjects or control population has been normalized.

58. The method of claim 56, wherein normalization comprises comparing the amount of the CYP gene amplified product to a determined amount of amplified product for a control gene.

59. The method of claim 58, wherein the comparing step comprises generating a ratio of the determined amount of CYP gene amplified product to the determined amount of amplified product for a control gene.

60. The method of claim 59, wherein selecting a therapeutic regimen comprises comparing the ratio of CYP gene amplified product to control gene amplified product to the same ratio from control subjects or a control population.

61. The method of claim 55, wherein the biological sample is a whole blood sample.

62. A method for determining a patient's therapeutic regimen for a drug metabolized by CYP2D6 comprising:
(a) obtaining a whole blood sample from the patient;
(b) isolating and reverse transcribing RNA from the sample to obtain cDNA;
(c) subjecting the cDNA to amplification using a pair of oligonucleotide primers capable of amplifying a region of the CYP2D6 gene to obtain an amplified sample;
(d) determining the amount of CYP2D6 cDNA in the amplified sample;
(e) generating a ratio of the determined amount of CYP2D6 cDNA from step (d) to a determined amount of cDNA for a control gene; and
(f) selecting a therapeutic regimen based on the generated ratio.

63. The method of claim 62, wherein selecting a therapeutic regimen comprises comparing the generated ratio from the patient to the same ratios obtained from control subjects or a control population.

64. A method for detecting SNPs of NAT1 gene in a sample comprising:
(a) isolating DNA from the sample;
(b) subjecting the DNA to amplification;
(c) hybridizing the amplified DNA sample with electronically arrayed oligonucleotides that hybridize with NAT1 SNPs.

65. The method of claim 64, wherein the method further comprises the step of detecting the presence of fluorescence from hybridized pairs.

66. The method of claim 64, wherein the sample is whole blood.

67. The method of claim 65, wherein the detection is by microarray scanner.

68. The method of claim 64, wherein the NAT1 SNPs are selected from FIG. 13.

69. A method for detecting SNPs of NAT2 gene in a sample comprising:
(a) isolating DNA from the sample;
(b) subjecting the DNA to amplification;
(c) hybridizing the amplified DNA sample with electronically arrayed oligonucleotides that hybridize with NAT2 SNPs.

70. The method of claim 69, wherein the method further comprises the step of detecting the presence of fluorescence from hybridized pairs.

71. The method of claim 70, wherein the detection is by microarray scanner.

72. The method of claim 69, wherein the NAT2 SNPs are selected from FIG. 14.

73. The method of claim 69, wherein the sample is whole blood.

74. A method of determining a patient's therapeutic regimen for a drug metabolized by NAT1 and/or NAT2 comprising:
(a) obtaining a sample from the patient;
(b) obtaining DNA from the sample;
(c) subjecting the DNA to amplification;
(d) hybridizing the amplified DNA sample with labeled probes for SNPs of the NAT1 gene and or the SNPs of the NAT2 gene; and
(e) determining the patient's therapeutic regimen based on the presence or absence of SNPs of the NAT1 gene and/or of the NAT2 gene in the amplified DNA sample.

75. A method of determining a patient's therapeutic regimen for a drug metabolized by CYP2D6 comprising:
(a) obtaining a sample from the patient;
(b) isolating DNA from the sample;
(c) subjecting the DNA to amplification;
(d) subjecting the amplified sample to microspheres labeled with oligonucleotide probes for CYP2D6 SNPs; and
(e) determining the patient's therapeutic regimen based on the presence or absence of SNPs of the CYP2D6 gene in the amplified DNA sample.

76. The method of claim 75, wherein the SNPs comprise one or more of the CYP2D6 SNPs listed in FIG. 12.

77. The method of claim 75, wherein the sample is a whole blood sample.

78. The method of claim 75, wherein the microspheres are labeled with the oligonucleotide probes using universally tagged primers.

79. The method of claim 75, wherein the microspheres are labeled with the oligonucleotide probes using a unilinker.
